(19) United States
(12) Patent Application Publication
(10) Pub. No.: US 2023/0183694 A1
(43) Pub. Date: Jun. 15, 2023

(54) ANTAGONIST OF PCSK9

(71) Applicant: ARGONAUTE RNA LIMITED, Bristol (GB)

(72) Inventors: Daniel Mitchell, Bristol (GB); Michael Khan, Bristol (GB)

(73) Assignee: ARGONAUTE RNA LIMITED, Bristol (GB)

(21) Appl. No.: 17/908,225

(22) PCT Filed: Mar. 15, 2021
(86) PCT No.: PCT/EP2021/056540

§ 371 Date: Aug. 30, 2022

(30) Foreign Application Priority Data
Mar. 16, 2020 (GB) 2003756.0
Jul. 3, 2020 (GB) 2010276.0
Sep. 7, 2020 (GB) 2013998.6
Dec. 23, 2020 (GB) 2020553.0

(51) Int. Cl. C12N 15/113; A61P 3/06

(52) U.S. Cl. CPC C12N 15/113; A61P 3/06; C12N 2310/11; C12N 2310/531

(57) ABSTRACT

This disclosure relates to a nucleic acid comprising a double stranded RNA molecule comprising sense and antisense strands and further comprising a single stranded DNA molecule covalently linked to the 3′ end of either the sense or antisense RNA part of the molecule wherein the double stranded inhibitory RNA targets proprotein convertase subtilisin kexin type 9 (PCSK9); pharmaceutical compositions comprising said nucleic acid molecule and methods for the treatment of diseases associated with increased levels of PCSK9, for example hypercholesterolemia and cardiovascular disease.

### CROSS REFERENCE TO RELATED APPLICATIONS

This is the U.S. National Stage of International Application No. PCT/EP2021/056540, filed Mar. 15, 2021, which was published in English under PCT Article 21(2), which in turn claims the benefit of Great Britain Application No. 2003756.0, filed Mar. 16, 2020, Great Britain Application No. 2010276.0, filed Jul. 3, 2020, Great Britain Application No. 2013998.6, filed Sep. 7, 2020, and Great Britain Application No. 2020553.0, filed Dec. 23, 2020.

### SEQUENCE LISTING

The Sequence Listing is submitted as an ASCII text file, named 108750-01_5 T25, created on Feb. 3, 2023, 33,147 bytes, which is herein incorporated by reference.

### FIELD OF THE DISCLOSURE

This disclosure relates to a nucleic acid comprising a double stranded RNA molecule comprising sense and antisense strands and further comprising a single stranded DNA molecule covalently linked to the 3′ end of either the sense or antisense RNA part of the molecule wherein the double stranded inhibitory RNA targets proprotein convertase subtilisin kexin type 9 (PCSK9); pharmaceutical compositions comprising said nucleic acid molecule and methods for the treatment of diseases associated with increased levels of PCSK9, for example hypercholesterolemia and cardiovascular disease.

### BACKGROUND TO THE DISCLOSURE

Cardiovascular disease associated with hypercholesterolemia, for example ischaemic cardiovascular disease is a common condition and results in heart disease and a high incidence of death and morbidity and can be a consequence of poor diet, obesity or an inherited dysfunctional gene. For example, PSCK9 is associated with familial hypercholesterolemia. Cholesterol is essential for membrane biogenesis in animal cells. The lack of water solubility means that cholesterol is transported around the body in association with lipoproteins. Apolipoproteins form together with phospholipids, cholesterol and lipids lipoproteins which facilitate the transport of lipids such as cholesterol through the bloodstream to the different parts of the body. Lipoproteins are classified according to size and can form HDL (High-density lipoprotein), LDL (Low-density lipoprotein), IDL (intermediate-density lipoprotein), VLDL (very low-density lipoprotein) and ULDL (ultra-low-density lipoprotein) lipoproteins.

Lipoproteins change composition throughout their circulation comprising different ratios of apolipoproteins A (ApoA), B (ApoB), C (ApoC), D(ApoD) or E (ApoE), triglycerides, cholesterol and phospholipids. ApoB is the main apolipoprotein of ULDL and LDL and has two isoforms apoB-48 and apoB-100. Both ApoB isoforms are encoded by one single gene and wherein the shorter ApoB-48 gene is produced after RNA editing of the ApoB-100 transcript at residue 2180 resulting in the creation of a stop codon. ApoB-100 is the main structural protein of LDL and serves as a ligand for a cell receptor which allows transport of, for example, cholesterol into a cell.

Familial hypercholesterolemia is an orphan disease and results from elevated levels of LDL cholesterol (LDL-C) in the blood. The disease is an autosomal dominant disorder with both the heterozygous (350-550 mg/dL LDL-C) and homozygous (650-1000 mg/dL LDL-C) states resulting in elevated LDL-C. The heterozygous form of familial hypercholesterolemia is around 1:500 of the population. The homozygous state is much rarer and is approximately 1:1,000,000. The normal levels of LDL-C are in the region 130 mg/dL.

Hypercholesterolemia is particularly acute in paediatric patients which if not diagnosed early can result in accelerated coronary heart disease and premature death. If diagnosed and treated early the child can have a normal life expectancy. In adults, high LDL-C, either because of mutation or other factors, is directly associated with increased risk of atherosclerosis which can lead to coronary artery disease, stroke or kidney problems. Lowering levels of LDL-C is known to reduce the risk of atherosclerosis and associated conditions. LDL-C levels can be lowered initially by administration of statins which block the de novo synthesis of cholesterol by inhibiting the HMG-CoA reductase. Some subjects can benefit from combination therapy which combines a statin with other therapeutic agents such as ezetimibe, colestipol or nicotinic acid. However, expression and synthesis of HMG-CoA reductase adapts in response to the statin inhibition and increases over time, thus the beneficial effects are only temporary or limited after statin resistance is established.

There is therefore a desire to identify alternative therapies that can be used alone or in combination with existing therapeutic approaches to control cardiovascular disease because of elevated LDL-C.

A technique to specifically ablate gene function is through the introduction of double stranded inhibitory RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically, but not exclusively, derived from exons of the gene which is to be ablated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

PCSK9 is a known target for therapeutic intervention in the treatment of hypercholesterolemia, cardiovascular disease and associated conditions. For example, WO2008/011431 discloses the use of short interfering nucleic acids that target PCSK9 expression and their use in the treatment of diseases and conditions such as hyperlipidaemia, hypercholesterolemia, cardiovascular disease, atherosclerosis and hypertension. Furthermore, WO2012058693 similarly discloses siRNA designed to silence PCSK9 gene expression in the treatment of pathologies associated with PCSK9 expression. Other disclosures that concern the inhibition of PCSK9 expression include U.S. Ser. No. 12/478,452, WO2009/134487 and WO2007/134487.

This disclosure relates to a nucleic acid molecule comprising a double stranded inhibitory RNA that is modified by the inclusion of a short DNA part linked to the 3′ end of either the sense or antisense inhibitory RNA and which forms a hairpin structure and is designed with reference to the nucleotide sequence encoding PCSK9. U.S. Pat. No. 8,067,572, which is incorporated by reference in its entirety, discloses examples of said nucleic acid molecules. The double stranded inhibitory RNA uses solely or predominantly natural nucleotides and does not require modified nucleotides or sugars that prior art double stranded RNA molecules typically utilise to improve pharmacodynamics and pharmacokinetics.

The disclosed double stranded inhibitory RNAs have activity in silencing PCSK9 with potentially fewer side effects.

### STATEMENTS OF THE INVENTION

According to an aspect of the invention there is provided a nucleic acid molecule comprising a first part that comprises a double stranded inhibitory ribonucleic acid (RNA) molecule comprising a sense strand and an antisense strand of at least part of the human PCSK9 nucleotide sequence; and

a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, wherein said single stranded DNA molecule comprises a nucleotide sequence that is adapted over at least part of its length to anneal by complementary base pairing to a part of said single stranded DNA to form a double stranded DNA structure comprising a double stranded stem domain and a single stranded loop domain.

According to an aspect of the invention there is provided a nucleic acid molecule comprising a first part that comprises a double stranded inhibitory ribonucleic acid (RNA) molecule comprising a sense strand and an antisense strand of at least part of the human PCSK9 nucleotide sequence or polymorphic sequence variant thereof; and

a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, wherein said single stranded DNA molecule comprises a nucleotide sequence that is adapted over at least part of its length to anneal by complementary base pairing to a part of said single stranded DNA to form a double stranded DNA structure comprising a double stranded stem domain and a single stranded loop domain.

A “polymorphic sequence variant” is a sequence that varies by one, two, three or more nucleotides. Preferably said double stranded inhibitory RNA molecule comprises natural nucleotide bases.

In a preferred embodiment of the invention wherein the 5′ end of said single stranded DNA molecule is covalently linked to the 3′ end of the sense strand of the double stranded inhibitory RNA molecule.

In a preferred embodiment of the invention wherein the 5′ end of said single stranded DNA molecule is covalently linked to the 3′ end of the antisense strand of the double stranded inhibitory RNA molecule.

In a preferred embodiment of the invention said loop domain comprises a region comprising the nucleotide sequence GNA or GNNA, wherein each N independently represents guanine (G), thymidine (T), adenine (A), or cytosine (C).

In a preferred embodiment of the invention said loop domain comprises G and C nucleotide bases.

In an alternative embodiment of the invention said loop domain comprises the nucleotide sequence GCGAAGC.

In a preferred embodiment of the invention said single stranded DNA molecule comprises the nucleotide sequence TCACCTCATCCCGCGAAGC (SEQ ID NO: 133).

In a preferred embodiment of the invention said double stranded inhibitory RNA molecule is between 10 and 40 nucleotide base pairs in length.

In a preferred embodiment of the invention said double stranded inhibitory RNA molecule is between 18 and 29 nucleotide base pairs in length.

In a preferred embodiment of the invention said double stranded inhibitory RNA molecule is between 19 and 23 nucleotide base pairs in length

In a preferred embodiment of the invention said double stranded inhibitory RNA molecule is 21 nucleotide base pairs in length.

Inhibitory RNA molecules comprise natural nucleotide bases that do not require chemical modification. Moreover, in some embodiments of the invention, wherein the crook DNA molecule is linked to the 3′ end of the sense strand of said double stranded inhibitory RNA, the antisense strand is optionally provided with at least a two-nucleotide base overhang sequence. The two-nucleotide overhang sequence can correspond to nucleotides encoded by the target e.g., PCSK9 or are non-encoding. The two-nucleotide overhang can be two nucleotides of any sequence and in any order, for example UU, AA, UA, AU, GG, CC, GC, CG, UG, GU, UC, CU.

In a preferred embodiment of the invention said double stranded inhibitory RNA molecule has at least 70% inhibition of PCSK9 mRNA expression as measured in an in vitro cell culture method of RNA silencing as herein disclosed.

In a preferred embodiment of the invention said in vitro cell culture method is silencing of PCSK9 expression in a HEPG2 cell.

Preferably, said double stranded inhibitory RNA molecule has at least 70%, 80%, 85% or 90% inhibition of PCSK9 mRNA expression.

In a preferred embodiment of the invention said double stranded inhibitory RNA molecule comprises or consists of between 18 and 29 contiguous nucleotides of the sense nucleotide sequence set forth in SEQ ID NO: 134.

Preferably, said double stranded inhibitory RNA molecule comprises or consists of 21 contiguous nucleotide bases pairs of the sense nucleotide sequence set forth in SEQ ID NO: 134.

In a preferred embodiment of the invention said double stranded inhibitory RNA molecule comprises a sense nucleotide sequence selected from the group consisting of: SEQ ID NO: 8, 1, 2, 3, 4, 5, 6, 7, 9 or 10.

In a preferred embodiment of the invention said double stranded inhibitory RNA molecule comprises an antisense nucleotide sequence selected from the group consisting of: SEQ ID NO: 18, 11, 12, 13, 14, 15, 16, 17, 19 or 20.

In an alternative preferred embodiment of the invention said double stranded inhibitory RNA molecule comprises a sense nucleotide sequence selected from the group consisting of: SEQ ID NO: 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 and 76.

In an alternative preferred embodiment of the invention said double stranded inhibitory RNA molecule comprises an antisense nucleotide sequence selected from the group consisting of: SEQ ID NO: 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131 and 132.

In a preferred embodiment of the invention said double stranded inhibitory RNA molecule comprises a sense strand comprising SEQ ID NO: 8 and an antisense strand comprising SEQ ID NO: 18.

In a preferred embodiment of the invention said single stranded DNA molecule is covalently linked to a sense strand comprising SEQ ID NO: 8.

In an alternative preferred embodiment of the invention said single stranded DNA molecule is covalently linked to an antisense strand comprising SEQ ID NO: 18.

In a preferred embodiment of the invention said double stranded inhibitory RNA molecule comprises a sense strand comprising SEQ ID NO: 9 and an antisense strand comprising SEQ ID NO: 19.

In a preferred embodiment of the invention said single stranded DNA molecule is covalently linked to a sense strand comprising SEQ ID NO: 9.

In an alternative preferred embodiment of the invention said single stranded DNA molecule is covalently linked to an antisense strand comprising SEQ ID NO: 19.

In a preferred embodiment of the invention said double stranded inhibitory RNA molecule comprises a sense strand comprising SEQ ID NO: 10 and an antisense strand comprising SEQ ID NO: 20.

In a preferred embodiment of the invention said single stranded DNA molecule is covalently linked to a sense strand comprising SEQ ID NO: 10.

In an alternative preferred embodiment of the invention said single stranded DNA molecule is covalently linked to an antisense strand comprising SEQ ID NO: 20.

In a preferred embodiment of the invention said double stranded inhibitory RNA molecule comprises a sense strand comprising SEQ ID NO: 135 and an antisense strand comprising SEQ ID NO: 136.

U.S. Pat. No. 10,851,3777 and US2018/104360, each of which is incorporated by reference in their entirety disclose siRNAs that target PCSK9. SEQ ID NO: 135 and SEQ ID NO: 136 are specifically claimed and are extensively modified using unnatural nucleotide bases. This siRNA is referred to as “inclisiran”. The present disclosure has adapted SEQ ID NO: 135 and 136 by the provision of the DNA part of the claimed nucleic acid molecule to either sequence to provide an alternative siRNA that uses natural nucleotide bases.

In a preferred embodiment of the invention said nucleic acid molecule is covalently linked to N-acetylgalactosamine.

In a preferred embodiment of the invention N-acetylgalactosamine is linked, directly or indirectly to the DNA part of said nucleic acid molecule via a terminal 3′ end of the DNA part.

In a preferred embodiment of the invention N-acetylgalactosamine is linked indirectly to the DNA part of said nucleic acid molecule via a cleavable linker, for example a thiol containing cleavable linker.

Chemistries that link ligands to oligonucleotides are known in the art. For, example the linkage of ligands such as N-acetylgalactosamine, to oligonucleotides is described in Johannes Winkler, Ther. Deliv. (2013) 4(7), 791-809 which is incorporated by reference in its entirety; see below in table 1:

TABLE 1

| | |
|---|---|
| 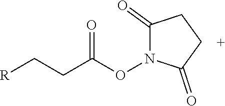 | A |
| 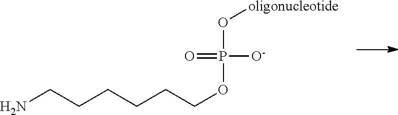 | |
| 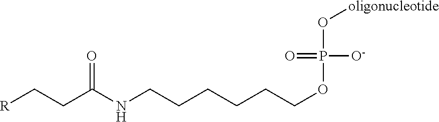 | |
| 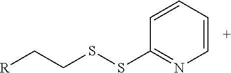 | B |
| 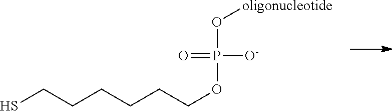 | |
| 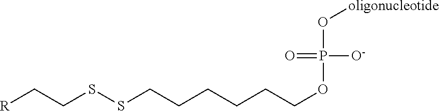 | |
| 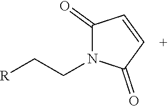 | C |
| 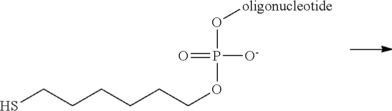 | |
| 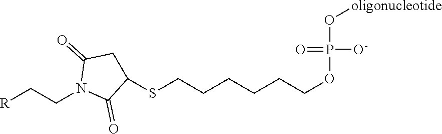 | |
| 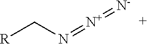 | D |
| 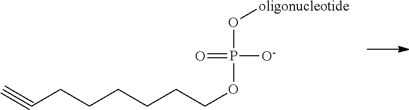 | |
| 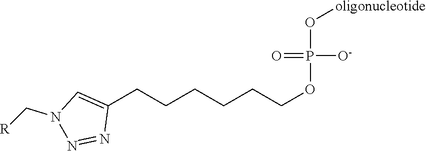 | |
| 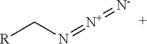 | E |
| 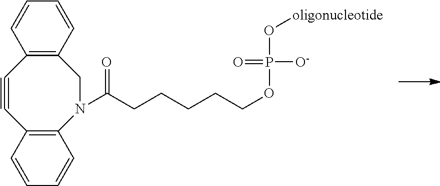 | |
| 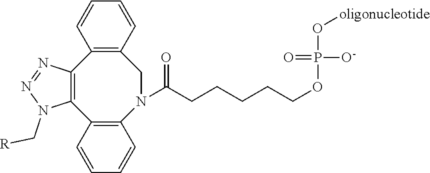 | |

A: Amide linkage formed via an active ester B: Disulfide linkage formed via pyridyldithiol activated ligand C: Thiol-maleimide coupling D: Copper catalyzed click chemistry coupling between an azide and alkyne E: Copper free click chemistry coupling between dibenzo-cyclooctyne and an azide.

Furthermore, alternative coupling chemistries to link ligands such as N-acetylgalactosamine, to oligonucleotides are disclosed in Yashveer Singh, Pierre Murat, Eric Defrancq, Chem. Soc. Rev., 2010, 39, 2054-2070 which is incorporated by reference in its entirety; see table 2 below:

TABLE 2

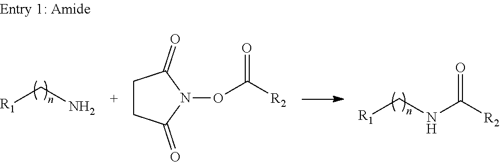

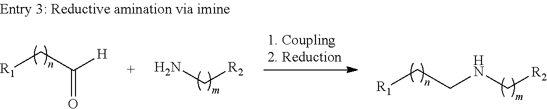

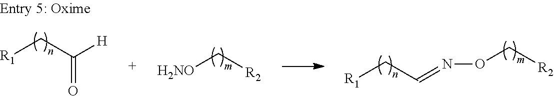

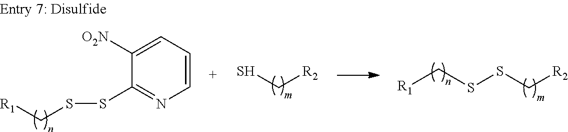

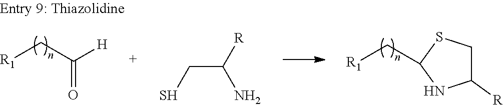

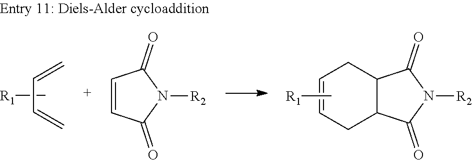

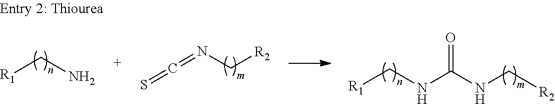

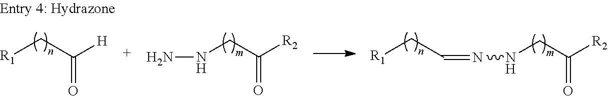

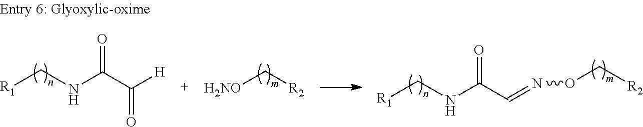

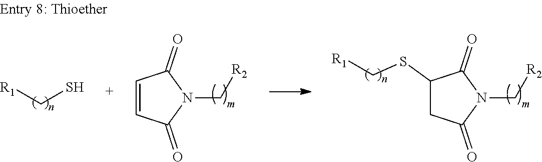

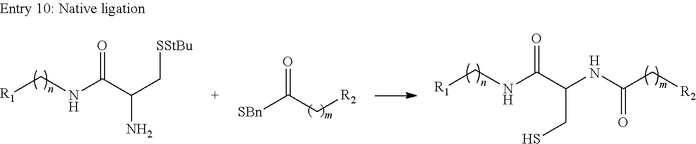

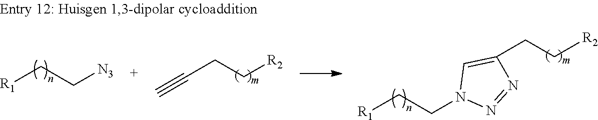

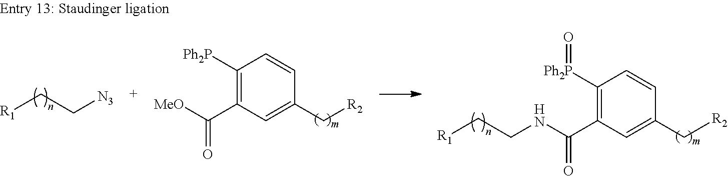

R₁ = Oligonucleoide
R₂ = Reporter moiety

In a further alternative embodiment of the invention N-acetylgalactosamine is linked to either the antisense part of said inhibitory RNA or the sense part of said inhibitory RNA.

In a preferred embodiment of the invention said nucleic acid molecule is covalently linked to a molecule comprising the structure:

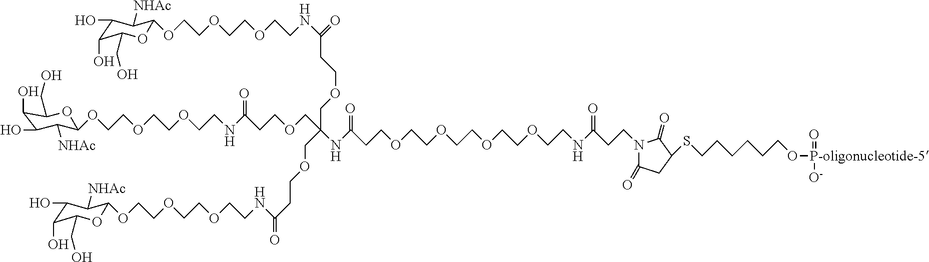

In an alternative preferred embodiment of the invention said nucleic acid molecule is covalently linked to a molecule comprising N-acetylgalactosamine 4-sulfate.

According to a further aspect of the invention there is provided a pharmaceutical composition comprising at least one nucleic acid molecule according to the invention.

In a preferred embodiment of the invention said composition further includes a pharmaceutical carrier and/or excipient.

In a preferred embodiment of the invention said pharmaceutical composition comprises at least one further, different, therapeutic agent. When administered the compositions of the present invention are administered in pharmaceutically acceptable preparations. Such preparations may routinely contain pharmaceutically acceptable concentrations of salt, buffering agents, preservatives, compatible carriers and optionally other therapeutic agents, such as cholesterol lowering agents, which can be administered separately from the nucleic acid molecule according to the invention or in a combined preparation if a combination is compatible.

The combination of a nucleic acid according to the invention and the other, different therapeutic agent is administered as simultaneous, sequential or temporally separate dosages.

The therapeutics of the invention can be administered by any conventional route, including injection or by gradual infusion over time. The administration may, for example, be oral, intravenous, intraperitoneal, intramuscular, intracavity, subcutaneous, transdermal or transepithelial.

The compositions of the invention are administered in effective amounts. An “effective amount” is that amount of a composition that alone, or together with further doses, produces the desired response. In the case of treating a disease, such as cardiovascular disease, the desired response is inhibiting or reversing the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a nucleic acid molecule according to the invention for producing the desired response in a unit of weight or volume suitable for administration to a patient. The response can, for example, be measured by determining regression of cardiovascular disease and decrease of disease symptoms etc.

The doses of the nucleic acid molecule according to the invention administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. It will be apparent that the method of detection of the nucleic acid according to the invention facilitates the determination of an appropriate dosage for a subject in need of treatment.

In general, doses of the nucleic acid molecules herein disclosed of between 1 nM-1 μM generally will be formulated and administered according to standard procedures. Preferably doses can range from 1 nM-500 nM, 5 nM-200 nM, 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a nonhuman primate, cow, horse, pig, sheep, goat, dog, cat or rodent.

When administered, the pharmaceutical preparations of the invention are applied in pharmaceutically acceptable amounts and in pharmaceutically acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents e.g. statins. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

Compositions may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “pharmaceutically acceptable carrier” in this context denotes an organic or inorganic ingredient, natural or synthetic, with which the active ingredient is combined to facilitate, for example, solubility and/or stability. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including acetic acid in a salt; citric acid in a salt; boric acid in a salt; and phosphoric acid in a salt. The pharmaceutical compositions also may contain, optionally, suitable preservatives.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product. Compositions suitable for oral administration may be presented as discrete units, such as capsules, tablets, lozenges, each containing a predetermined amount of the active compound.

Compositions suitable for parenteral administration conveniently comprise a sterile aqueous or non-aqueous preparation of nucleic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In a preferred embodiment of the invention said further therapeutic agent is a statin.

Statins are commonly used to control cholesterol levels in subjects that have elevated LDL-C. Statins are effective in preventing and treating those subjects that are susceptible and those that have cardiovascular disease. The typical dosage of a statin is in the region 5 to 80 mg but this is dependent on the statin and the desired level of reduction of LDL-C required for the subject suffering from high LDL-C. However, expression and synthesis of HMG-CoA reductase, the target for statins, adapts in response to statin administration thus the beneficial effects of statin therapy are only temporary or limited after statin resistance is established.

Preferably said statin is selected from the group consisting of atorvastatin, fluvastatin, lovastatin, pitvastatin, pravastatin, rosuvastatin and simvastatin.

In a preferred embodiment of the invention said further therapeutic agent is ezetimibe. Optionally, ezetimibe is combined with at least one statin, for example simvastatin.

In an alternative preferred embodiment of the invention said further therapeutic agent is selected from the group consisting of fibrates, nicotinic acid, cholestyramine.

In a further alternative preferred embodiment of the invention said further therapeutic agent is a therapeutic antibody, for example, evolocumab, bococizumab or alirocumab.

According to a further aspect of the invention there is provided a nucleic acid molecule according to the invention or a pharmaceutical composition according to the invention for use in the treatment or prevention of a subject that has or is predisposed to hypercholesterolemia or a disease associated with hypercholesterolemia.

In a preferred embodiment of the invention said subject is a paediatric subject.

A paediatric subject includes neonates (0-28 days old), infants (1-24 months old), young children (2-6 years old) prepubescent (7-14 years old) and pubescent children (14-18 years old).

In an alternative preferred embodiment of the invention said subject is an adult subject.

In a preferred embodiment of the invention the hypercholesterolemia is familial hypercholesterolemia.

In a preferred embodiment of the invention familial hypercholesterolemia is associated with elevated levels of PCSK9 expression.

In a preferred embodiment of the invention said subject is resistant to statin therapy.

In a preferred embodiment of the invention said disease associated with hypercholesterolemia is selected from the group consisting of: stroke prevention, hyperlipidaemia, cardiovascular disease, atherosclerosis, coronary heart disease, cerebrovascular disease, peripheral arterial disease, hypertension, metabolic syndrome, type II diabetes, non-alcoholic fatty acid liver disease and non-alcoholic steatohepatitis.

According to a further aspect of the invention there is provided a method to treat a subject that has or is predisposed to hypercholesterolemia comprising administering an effective dose of a nucleic acid or a pharmaceutical composition according to the invention thereby treating or preventing hypercholesterolemia or a disease associated with hypercholesterolemia.

In a preferred method of the invention said subject is a paediatric subject.

In an alternative preferred method of the invention said subject is an adult subject.

In a preferred method of the invention the hypercholesterolemia is familial hypercholesterolemia.

In a preferred method of the invention familial hypercholesterolemia is associated with elevated levels of proprotein convertase subtilisin kexin type 9 (PCSK9) expression.

In a preferred method of the invention said subject is resistant to statin therapy.

In a preferred method of the invention said disease associated with hypercholesterolemia is selected from the group consisting of: stroke prevention, hyperlipidaemia, cardiovascular disease, atherosclerosis, coronary heart disease, cerebrovascular disease, peripheral arterial disease, hypertension, metabolic syndrome, type II diabetes, non-alcoholic fatty acid liver disease and non-alcoholic steatohepatitis.

According to a further aspect of the invention there is provided a diagnostic method and treatment regimen for hypercholesterolemia associated with elevated PCSK9 comprising:
i) obtaining a biological sample from a subject suspected of having or having hypercholesterolemia;
ii) contacting the sample with an antibody, or antibody fragment, specific for a PSCK9 polypeptide;
iii) determining the concentration of said PCSK9 and LDL-C in said biological sample; and
iv) administering a nucleic acid molecule or pharmaceutical composition according to the invention if the LDL-C concentration is greater than 350 mg/dL.

Typically, in familial hypercholesterolemia disease the levels of LDL-C are 350-550 mg/dL in subjects that are heterozygous for a selected mutation and 650-1000 mg/dL in those subjects carrying a homozygous mutation. The normal levels of LDL-C are in the region 130 mg/dL.

Throughout the description and claims of this specification, the words “comprise” and “contain” and variations of the words, for example “comprising” and “comprises”, means “including but not limited to” and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by example only and with reference to the following figures:

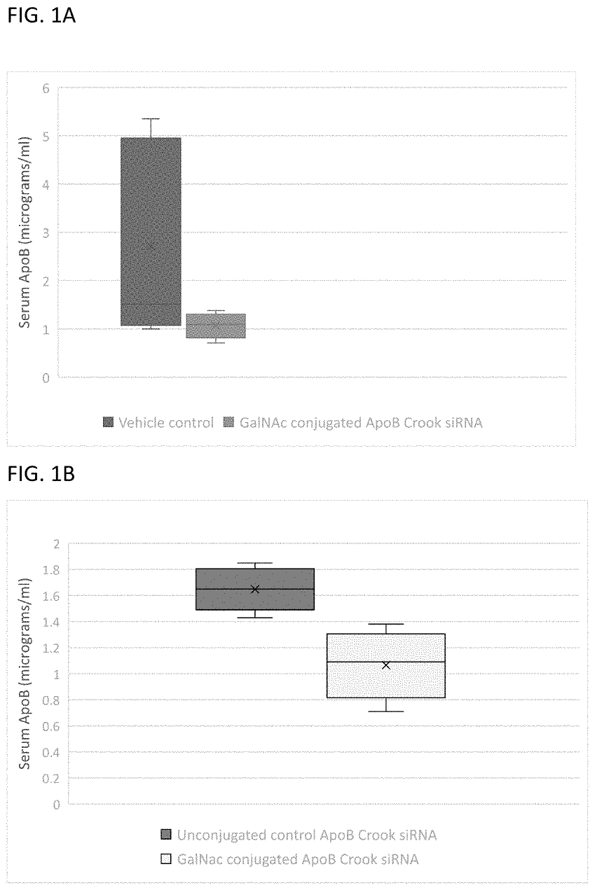

FIGS. 1A and 1B. Graphs illustrating in vivo Activity of GalNAc-conjugated Crook anti-mouse ApoB siRNA compared to controls. (FIG. 1A) Plasma ApoB levels (micrograms/ml) from five adult male wild-type C57BL/6 mice, were measured 96 hours following subcutaneous administration of GalNAc-conjugated ApoB Crook siRNA (one treatment group) and compared with the control treatment group administered with saline. Statistical analysis was applied using the two-tailed paired T test algorithm. Results show a substantive reduction in mean plasma ApoB levels in mice treated with GalNAc-conjugated Crook siRNA, compared to control. However, it just fails significance (p=0.08), most likely due to small sample size and variation in ApoB levels between control animals; (FIG. 1B) plasma ApoB levels (micrograms/ml) from five adult male wild-type C57BL/6 mice, were measured 96 hours following subcutaneous administration of GalNAc-conjugated ApoB Crook siRNA (one treatment group) and compared with the control treatment group, administered with siRNA construct unconjugated (without GalNAc) ApoB Crook siRNA. Statistical analysis was applied using the two-tailed paired T test algorithm. Results show a highly significant reduction in plasma ApoB levels in this GalNAc-conjugated Crook siRNA treatment group when compared to control unconjugated siRNA with Crook (P=0.00435832);

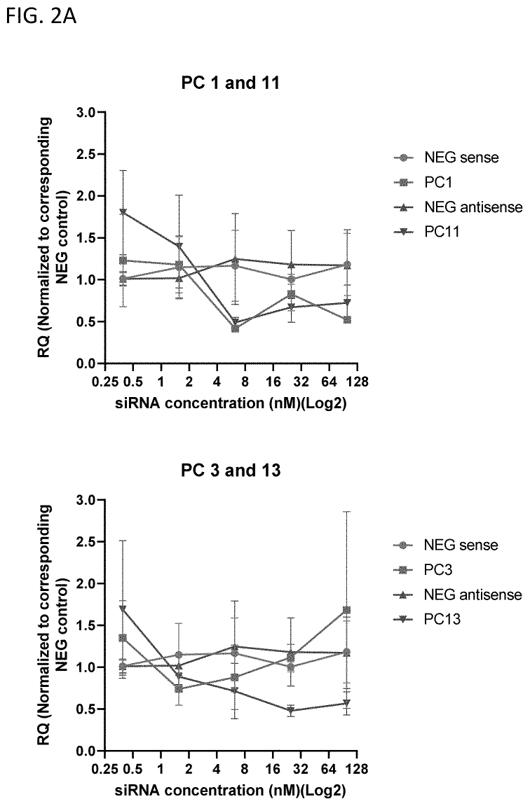

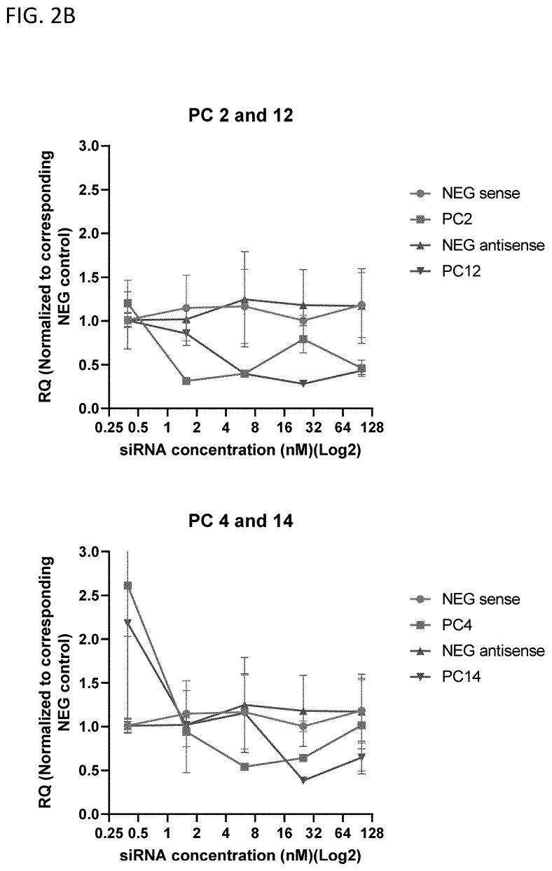

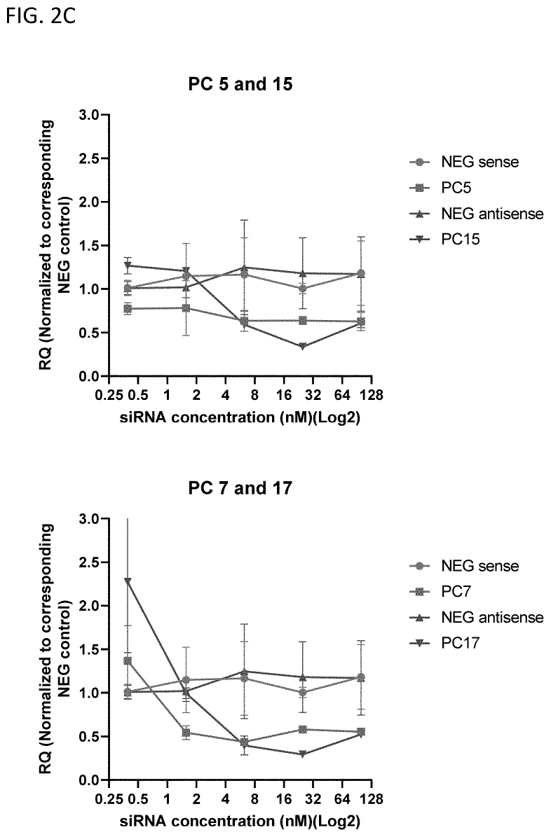

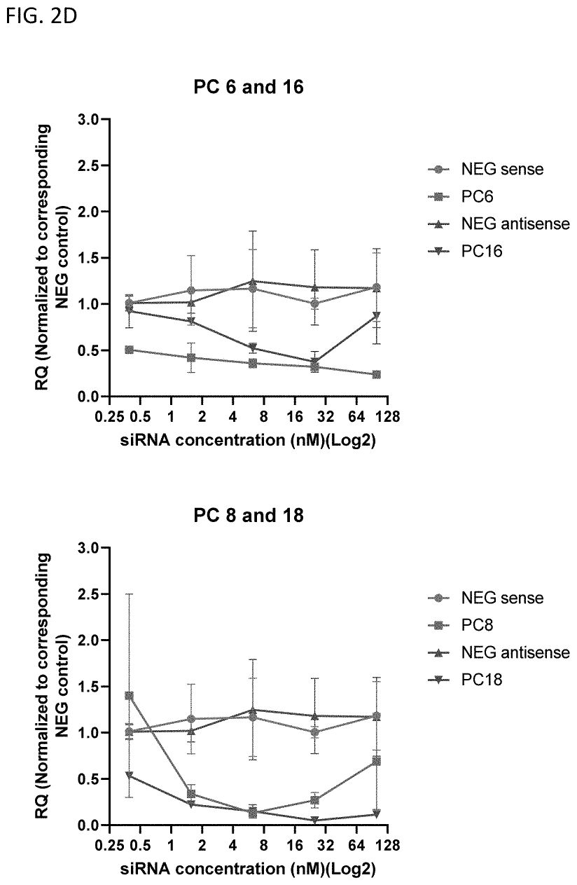

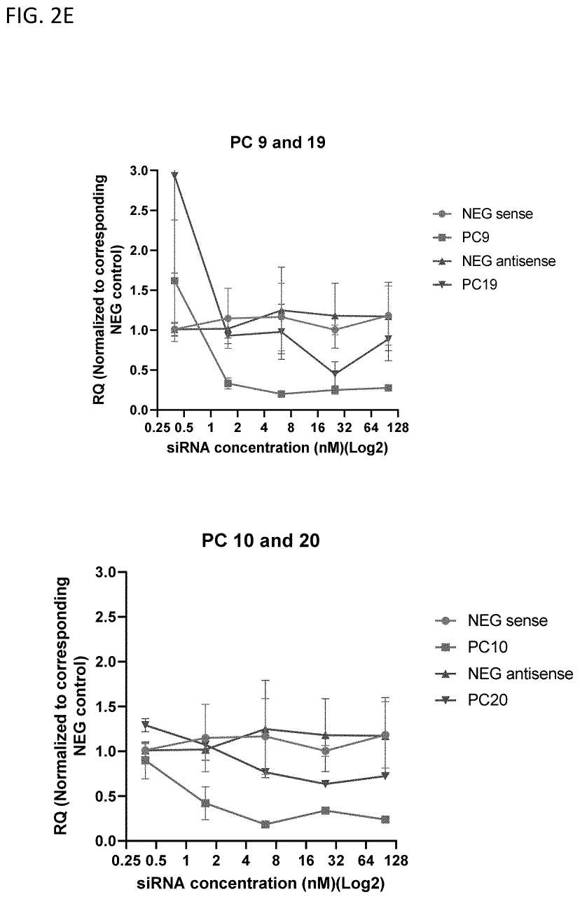

FIGS. 2A-2E illustrate an in vitro screen of 20 custom duplex Crook PCSK9 siRNAs (PC1-C20) listed in Table 1. Graphical presentation of data shows relative knock down of PCSK9 mRNA expression in HepG2 cells for each crook siRNA sense and antisense pair; PC1-C10 (sense strand); PC11-20 (antisense strand). Each crook siRNA molecule was reverse transfected into HepG2 cells (in quadruplicate) at five doses (100 nM, 25 nM, 6.25 nM, 1.56 nM and 0.39 nM) using the conditions identified in the assay development phase. 72 hours post transfection, cells were lysed and PCSK9 mRNA levels determined by duplex RT-qPCR. In order to calculate knockdown of PCSK9 (relative quantification; RQ) for each siRNA at each concentration, expression was first normalised to housekeeping reference gene GAPDH mRNA expression and then to the average PCSK9 expression across the five doses of the corresponding negative (NEG) control (crook Sense or Antisense); (FIG. 2A) Crook siRNAs (PC1 (SEQ ID NO 1)+PC11 (SEQ ID NO 11); PC3 (SEQ ID NO 3)+PC13 (SEQ ID NO 13); (FIG. 2B) PC2 (SEQ ID NO 2)+PC12 (SEQ ID NO 12)+; PC4 (SEQ ID NO 4)+PC14(SEQ ID NO 14)); (FIG. 2C) PC5+PC15 (SEQ ID NO 5+15); PC7+PC17(SEQ ID NO 7+17); (FIG. 2D) PC6+PC16 (SEQ ID NO 6+16); PC8+PC18 (SEQ ID NO 8+18); (FIG. 2E) (PC9+PC19 (SEQ ID NO 9+19); PC10+PC20 (SEQ ID NO 10+20); and

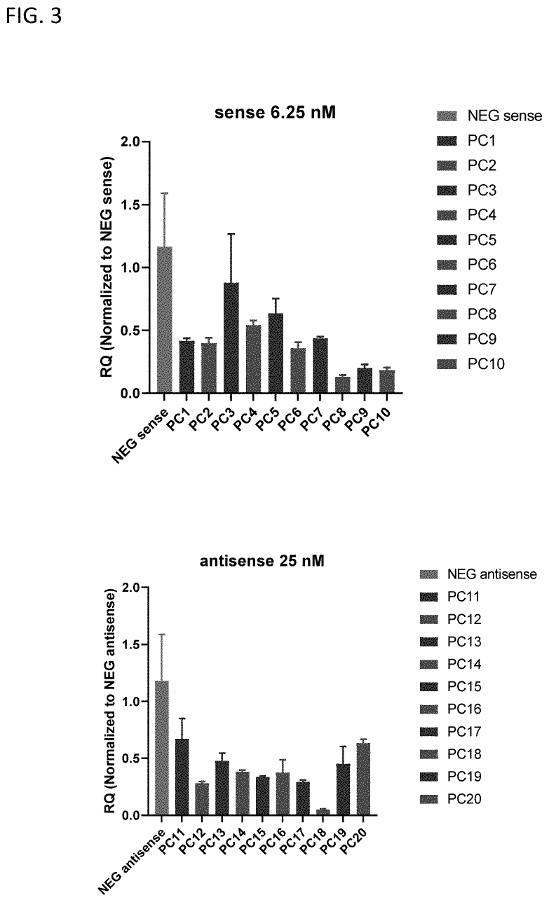

FIG. 3 presents a summary of PCSK9 knockdown in HepG2 cells of crook siRNAs at the optimal concentration of 6.25 nm or 25 nM sense (PC1-10) or antisense (PC11-20) respectively.

### Materials and Methods

PCSK9 siRNA In Vitro Screen Reverse Transfection and RT-qPCR Protocols

### 1. HepG2 Reverse Transfection

Custom duplex siRNAs synthesized by Horizon Discovery were resuspended in UltraPure DNase and RNase free water to generate a stock solution of 10 μM.
tock siRNAs were dispensed into 4×384-well assay plates (Greiner #781092). On each assay plate, 10 Custom siRNAs and 3 controls (POS PCSK9, NEG sense and NEG antisense) were dispensed to generate five-point four-fold dilution series from a top final concentration in the assay plate of 100 nM. ON TARGETplus Non-Targeting and PCSK9 siRNAs controls were dispensed to give a final concentration of 25 nM.
Lipofectamine RNAiMAX (ThermoFisher) was diluted in Optimem media before 10 μL of the Lipfectamine RNAiMAX:OptiMEM solution was added per well to the assay plate. The final volume of RNAiMAX per well was 0.08 μL.
The lipid-siRNA mix was incubated 30 min at room temperature.
HepG2 cells were diluted in assay media (MEM GlutaMAX (GIBCO) 10% FBS 1% Pen/Strep) before 4,000 HepG2 cells were seeded into each well of the assay plate in 40 μL volume. Quadruplicate technical replicates were seeded per assay condition.
The plates were incubated 72 h at 37° C., 5% CO₂ in a humidified atmosphere, prior to assessment of the cells.

### 2. PCSK9/GAPDH Duplex RT-qPCR

72 h post-transfection, cells were processed for RT-qPCR read-out using the Cells-to-CT 1-step TaqMan Kit (Invitrogen 4391851C). Briefly, cells were washed with 50 μl cold PBS and then lysed in 20 μl Lysis solution containing DNase I. After 5 min, lysis was stopped by addition of 2 μl STOP Solution for 2 min.
For the RT-qPCR analysis, 3 μl of lysate was dispensed per well into 384-well PCR plate as template in an 11 μl RT-qPCR reaction volume.
RT-qPCR was performed using the ThermoFisher TaqMan Fast Virus1-Step Master Mix (#4444434) with TaqMan probes for GAPDH (VIC #4448486) and

ApoB (FAM #4351368).
RT-qPCR was performed using a QuantStudio 6 thermocycling instrument (Applied BioSystems).
Relative quantification was determined using the ΔΔCT method, where GAPDH was used as internal control and expression changes normalized to the reference sample (either NEG sense or NEG antisense siRNA treated cells).

### Human PBMC Stimulation Assay (Judge et al. 2005, 2006)

A human PBMC assay are used to identify the potential of a variety of siRNA constructs to induce a cytokine storm. Primary PBMC from healthy donors (ATCC® PCS-800-011™) are seeded at a density of 2×10⁵ cells/well in 96-well microplates and cultured in triplicates in 200 μL RPMI 1640 medium with 10% FBS, 2 mM glutamine, 100 U/ml penicillin and 100 μg/ml streptomycin. siRNAs are added to cells at different concentrations (ranging 0.39-100 nM). The treatment groups include: 1) double-strand siRNA; 2) double-strand siRNA-crook on sense; 3) double-strand siRNA-crook on antisense; 4) double-strand immunostimulatory siRNA; 5) double-strand immunostimulatory siRNA-crook on sense; 6) double-strand immunostimulatory siRNA-crook on antisense; 7) vehicle; 8) untreated control and 9) lipopolysaccharide (LPS) at a concentration of 20-100 ng/mL. After adding the treatment, cells are incubated for 16-24 hours in a humidified 37° C., 5% CO2 incubator. The culture media is collected into 1.5 mL centrifuge tubes and centrifuged at a maximum speed for 5 minutes. Supernatants are collected into fresh tubes and either processed for cytokine analysis by ELISA or stored at −20° C.

TABLE 3
Controls for Monitoring Immune Stimulation by PBMCs

| Sequence | Sense (5′-3′) | Antisense (5′-3′) |
|---|---|---|
| Unmodified | CUAGACCUGUdTUUGCUUUUGU | ACAAAAGCAAAACAGGUCUAGAA |
| Inclisiran | (SEQ ID NO: 135) | (SEQ ID NO: 145) |
| ApoB-1 | GUCAUCACACUGAAUACCAAU | AUUGGUAUUCAGUGUGAUGACAC |
| | (SEQ ID NO: 137) | (SEQ ID NO: 138) |
| β-Gal | UUGAUGUGUUUAGUCGCUAUU | UAGCGACUAAACACAUCAAUU |
| | (SEQ ID NO: 139) | (SEQ ID NO: 140) |
| β-gal 728 | CUACACAAAUCAGCGAUUU | AAAUCGCUGAUUUGUGUAG |
| | (SEQ ID NO: 141) | (SEQ ID NO: 142) |
| Luc-siRNA | UAAGGCUAUGAAGAGAUACdTdT | AAGUAUCUCUUCAUAGCCUUA |
| | (SEQ ID NO: 143) | (SEQ ID NO: 144) |
| Poly(A:U) | Poly(A:U) - TLR3 Agonist - | |
| RNA | Polyadenylic - polyuridylic | |
| | acid (InvivoGen) | |

| | |
|---|---|
| Poly(I:C) | Synthetic dsRNA |
| LMW | (InvivoGen) |

### Cytokine ELISA

Cytokines are quantified using ELISA kits according to the manufacturer's instructions. The following ELISA kits are used to detect cytokine concentration in the cell culture media: human IFN-α (Invitrogen, Cat #BMS216), human IFN-γ (Invitrogen, Cat #EHIFNG), human IFN-β (Invitrogen, Cat #414101), human IL-6 (Invitrogen, Cat #BMS213HS) and TNF-α (Invitrogen; Catalog #KHC3011). An ELISA plate reader is used to measure the absorbance at a wavelength of 570 nm.

MTT Assay for Cell Viability (Abcam, MTT Assay Kit ab211091)

An MTT assay is used to determine cell viability after treatment of primary PBMC and HepG2 cells. Cells are seeded at a concentration of 2×10⁵ cells/well in a 96-well microplate with 100 μl of culture medium. Cells are treated with varying concentrations of siRNA constructs or appropriate controls and cultured for 16-48 hours at 37° C. and 5% CO₂. After treatment, microplates are centrifuged at 1,000 g for 5 minutes in a microplate-compatible centrifuge and media is carefully removed. Fifty μL of serum-free media and 50 μL of MTT Reagent are added into each well. Background control wells contain 50 μL MTT Reagent+50 μL cell culture media (w/o cells). The plate is incubated at 37° C. for 3 hours. After incubation, 150 μL of MTT Solvent is added into each well. The plate is wrapped in foil and incubated on an orbital shaker for 15 minutes. Absorbance is read at 590 nm. The amount of absorbance is proportional to cell number.

### Proteome Profiler Human Cytokine Array Kit (R&D System, ARY005B)

A cytokine array is performed for the simultaneous determination of selected human cytokines and chemokines in HepG2 cells and PBMC treated with siRNA constructs or appropriate controls. The assay uses a membrane-based antibody array to detect 36 human cytokines, chemokines, and acute phase proteins simultaneously. After treatment, the culture media of HepG2 and PBMC are collected and centrifuged to remove particulates. A range of 200-700 μL of cell culture supernatants is used for the assay. Cytokines are detected according to the manufacturer's instructions. Briefly, the nitrocellulose membrane spotted with different antibodies are incubated for one hour on a rocking platform with 2.0 mL of Array Buffer used as a block buffer. Each sample is prepared by adding 0.5 mL of Array Buffer and 15 μL of reconstituted Human Cytokine Array Detection Antibody Cocktail followed by 1 hour incubation at room temperature. Membranes are incubated overnight at 2-8° C. with sample/antibody mixtures followed by washings. Two mL of diluted Streptavidin-HRP is added to membranes and incubated for 30 minutes at room temperature. For cytokines visualization, membranes are incubated for 1 minute with 1 mL of the prepared Chemi Reagent Mix and placed in an autoradiography film cassette for 1-10 minutes. Spot intensity for each cytokine is quantified with the dot blot analyser from ImageJ and expressed as pixel intensity. Spot intensity will be normalized to cell number calculated using an MTT assay. Signals on different arrays are compared to determine the relative change in cytokine levels between samples.

### Stability Assay in Serum

It has been demonstrated that, the 3′-DNA mini-hairpin (Crook) conferred nuclease resistance to siRNA constructs in vitro and that resistance required the double-stranded RNA structure (Allison and Milner, 2014). For the stability assay, equivalent amounts of siRNA-crook and unmodified siRNAs targeting PCSK9 will be preincubated in culture medium containing 5% serum or no serum for 16 hours at 37° C. before transfection into HepG2 cells (see HepG2 transfection). The efficiency of both siRNAs will be then tested using qPCR to quantify the expression levels of the target gene (see PCR protocol).

In Vivo siRNA Activity in Mice.

Unconjugated and GalNAc conjugated versions of PCSK9 or ApoB Crook-siRNA were administered by IV and/or SC routes to investigate the relative plasma and tissue exposure. The rationale for dose selection was based on the following information published in the scientific literature:

The GalNAc conjugated siRNA is dosed subcutaneously at 2.0 mg/kg or 5 mg/kg which is expected to produce the required level of gene silencing where the ED₈₀ of structurally related siRNAs has been reported as 2.5 mg/kg (Soutschek et al., 2004). These structurally related siRNAs were tolerated up to 25 mg/kg, single administration, in the mouse (Soutschek et al., 2004).

The unconjugated version of the siRNA is administered at 50 mg/kg intravenously. This 10-fold increase in the IV compared to the SC dose is due to the unconjugated siRNA being less effective at targeting the liver. Additionally, it is reported by Soutschek et al (2004) that lower levels of RNA are measured in the liver following IV compared to SC administration. It is stated that slower release of the siRNA from the subcutaneous depot leads to prolonged exposure increasing the potential for receptor-ligand interactions and greater uptake into the tissue. Similar related siRNA has been well tolerated by mice at up to 50 mg/kg IV administered on 3 consecutive days (Nair et al. 2014). As a precaution a 15-minute observation period is left between dosing the 1^{st} animal IV to determine if the test substance causes any adverse effects before the remaining animals are dosed.

The mouse is the species of choice because it is used as one of the toxicology species in the safety testing of the test substance. The mouse also possesses a very similar metabolic physiology to humans in relation to the therapeutic target of the Crook-siRNA preparations (PCSK9 or ApoB). There is a considerable amount of published data available which are acceptable to the regulatory authorities for assessing the significance to man of data generated in this species.

### Animals

Sufficient C57BL/6 mice were obtained from an approved source to provide healthy male animals. Animals are in the target weight range of 20 to 30 g at dosing. Mice are uniquely numbered by tail marking. Numbers are allocated randomly. Cages are coded by cards giving information including study number and animal number. The study room is identified by a card giving information including room number and study number. On receipt, all animals were examined for external signs of ill health. Unhealthy animals where be excluded from the study. The animals were acclimatised for a minimum period of 5 days. Where practicable, without jeopardising the scientific integrity of the study, animals were handled as much as possible. A welfare inspection was performed before the start of dosing to ensure their suitability for the study.

The mice were kept in rooms thermostatically maintained at a temperature of 20 to 24° C., with a relative humidity of between 45 and 65%, and exposed to fluorescent light (nominal 12 hours) each day. Temperature and relative humidity are recorded on a daily basis. The facility is designed to give a minimum of 15 air-changes/hour. Except when in metabolism cages or recovering from surgery, mice were housed up to 5 per cage according to sex, in suitable solid floor cages, containing suitable bedding.

Cages conform to the ‘Code of Practice for the Housing and Care of Animals Bred, Supplied or Used for Scientific Purposes’ (Home Office, London, 2014). In order to enrich both the environment and the welfare of the animals, they were provided with wooden Aspen chew blocks and polycarbonate tunnels. The supplier provided certificates of analysis for each batch of blocks used. All animals will be allowed free access to 5LF2 EU Rodent Diet 14%. The diet supplier provided an analysis of the concentration of certain contaminants and some nutrients for each batch used. All animals were allowed free access to mains water from bottles attached to the cages. Periodic analysis of the mains supply is undertaken.

All procedures to be carried out on live animals as part of this study will be subject to provisions of United Kingdom National Law, the Animals (Scientific Procedures) Act 1986.

All animals were examined at the beginning and the end of the working day, to ensure that they are in good health. Any animal, which shows marked signs of ill health, were isolated. Moribund animals or those in danger of exceeding the severity limits imposed by the relevant Home Office Licence were killed.

### Crook GalNAc Conjugate Synthesis

The GalNAc component of the hepatocyte-targeting siRNA is a triantennary GalNAc cluster with a Cl 0 spacer and is conjugated to the 3′ terminus of either the sense or antisense strand of the siRNA via an aminopropanediol-based linker (described in Sharma et. al Bioconjugate Chem (2018) 29:2478-2488). For Crook siRNA molecules, GalNAc conjugation of the sense strand occurs at a deoxyribonucleotide terminus, and at the antisense at a ribonucleotide terminus.

GalNAc conjugated siRNAs are prepared using a protocol based on the solid phase method with GalNAc cluster-derivatised controlled pore glass support, as described by Nair et al J Amer Chem Soc (2014) 136:16958-16961.

### Structure of Final GalNAc Conjugate:

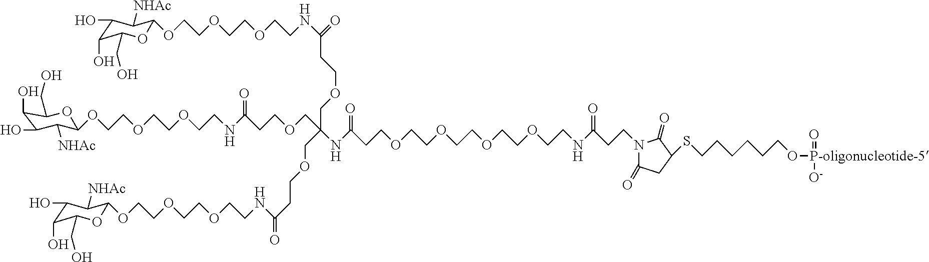

### Preparation of Formulations

Test substances were diluted in 0.9% saline to provided concentrations of 25 mg/mL and 0.6 mg/mL for the intravenous and subcutaneous doses of PCSK9 or ApoB Crook-siRNA GalNAc-unconjugated and conjugate respectively. The formulations were gently vortexed as appropriate until the test substances are fully dissolved. The resulting formulation(s) were assessed by visual inspection only and categorised accordingly:

(1) Clear solution

(2) Cloudy suspension, no particles visible

(3) Visible particles

After use, formulations were stored refrigerated nominally at 2-8° C.

### Dosing Details

### Apo B

Each animal received either a single intravenous dose of the ApoB Crook-siRNA GalNAc-unconjugated or a single subcutaneous dose ApoB Crook-siRNA GalNAc-conjugate. The intravenous dose was administered as a bolus into the lateral tail vein at a volume of 2 mL/kg. The subcutaneous dose was administered into the subcutaneous space at a volume of 5 mL/kg.

### PCSK9

For PCSK9, each animal received a single subcutaneous dose of the GalNAc conjugated PCSK9 crook siRNA and are monitored at 2 time points to determine PCSK9 silencing (96 hrs and 14 days). Samples are obtained either via tail bleed or cardiac puncture at conclusion.

For each of the PCSK9 crook siRNA

| | | |
|---|---|---|
| 10 mice | SC | GalNAc-conjugated PCSK9 crook-siRNA at 2 mg/kg |
| 10 mice | SC | GalNAc-conjugated PCSK9 crook-siRNA at 5 mg/kg |
| 10 mice | SC | GalNAc-conjugated crook unmodified inclisiran |
| | | sequence (SEQ ID NO: 135/136) |
| 10 mice | SC | saline control |

### Body Weights

As a minimum, body weights were recorded the day after arrival and before dose administration. Additional determinations were made, if required.

### Sample Storage

Samples were uniquely labelled with information including, where appropriate: study number; sample type; dose group; animal number/Debra code; (nominal) sampling time; storage conditions. Samples were stored at <−50° C.

### Blood Sampling

Serial blood samples of (nominally 100 μL, dependent on bodyweight) were collected by tail nick at the following times: 0, 48 96* hours post dose or 14 days. Animals were terminally anaesthetised using sodium pentobarbitone and a final sample (nominally 0.5 mL) was collected by cardiac puncture.

Blood samples were collected in to a K2EDTA microcapillary tube (tail nick) or a K2EDTA blood tube (cardiac puncture) and placed on ice until processed. Blood was centrifuged (1500 g, 10 min, 4° C.) to produce plasma for analysis. The bulk plasma was divided into two aliquots of equal volume. The residual blood cells were discarded. The acceptable time ranges for blood sample collections are summarised in the following table. Actual sampling times were recorded for all matrices.

TABLE 2

| Scheduled Collection | Acceptable |
|---|---|
| Time | Time Range |
| 0-15 minutes | ±1 minute |
| 16-30 minutes | ±2 minutes |
| 31-45 minutes | ±3 minutes |
| 46-60 minutes | ±4 minutes |
| 61 minutes-2 hours | ±5 minutes |
| 2 hours 1 minute-8 | ±10 minutes |
| hours | |
| 8 hours 1 minute-12 | ±15 minutes |
| hours | |
| 12 hours onwards | ±30 minutes |

Where a scheduled collection time is outside the acceptable range, the actual blood collection time was reported for inclusion in any subsequent PK analysis.

### Animal Fate

Animals were anaesthetised via an intraperitoneal injection of Sodium Pentobarbitone prior to terminal blood sampling and sacrificed by perfusion and exsanguination.

A full body perfusion was performed, all animals were flushed with Heparinised Saline Solution at a rate 4 ml/min for 5 minutes (approximately 20 mL total flush). Death was confirmed by the absence of breathing, heartbeat and blood flow. Animal carcasses were retained for tissue collection.

### Tissue Collection

The liver was removed from all animals and placed into a pre-weighed tube. The tissue samples were homogenised with 5 parts RNAlater to 1 part tissue using the UltraTurrax homogenisation probe. The following tissues were excised from animals in PCSK9 or ApoB treated groups and placed into a pre-weighed pot:
Spleen
Brain
Heart
Lung Lobes
Skin (Inguinal region ca. 25 mm²)

Following collection, the external surface of the tissues is rinsed with PBS and gently patted dry using a tissue. Tissues are initially placed on wet ice until weighed and then tissues were snap frozen on dry ice prior to storage. Tissues are stored at <−50° C. (nominally −80° C.).

### Immunoassays and Sample Analysis

Plasma PCSK9 or ApoB levels were measured via enzyme-linked immunosorbent assay (ELISA) using the commercial mouse PCSK9 or ApoB detection kit from Elabscience Biotechnology Inc. Plasma samples were stored at −80° C. prior to analysis, thawed on ice and centrifuged at 13,000 rpm for 5 minutes prior to aliquots being diluted in Assay Buffer and applied to the ELISA plate. The PCSK9 or ApoB assay kit uses a sandwich ELISA yielding a colorimetric readout, measured at OD450. Samples from each animal at specific time points (0 hours, 96 hours and 14 days) were assayed in duplicate and measurements were recorded as micrograms PCSK9 or ApoB per ml of plasma based on the standard curve reagents supplied with the kit. All data points were measured with a coefficient of variation <20%. Plasma PCSK9 or ApoB levels after the specified time-points following administration of GalNAc-conjugated PCSK9 or ApoB Crook siRNA were compared with the control treatment groups. Statistical analysis was applied using the two-tailed paired T test algorithm.

In addition, blood lipid profiles were obtained by measuring levels of ApoB, total cholesterol, HDL, triglycerides using standard assays.

### EXAMPLE 1

In vivo activity of GalNAc-conjugated Crook ApoB siRNA compared to control siRNA constructs. Plasma ApoB levels (micrograms/ml) from 5 mice in each treatment group, were used to calculate a mean ApoB value+/−standard error of the mean (SEM). Plasma ApoB levels after 96 hours following SC administration of GalNAc-conjugated Crook siRNA were compared to levels in mice receiving either control (i) vehicle saline, or (ii) unconjugated siRNA with Crook. Statistical analysis was applied using the two-tailed paired T test algorithm.

With reference to FIG. 1 (a), plasma ApoB levels (micrograms/ml) of mice 96 hours following treatment with GalNAc-conjugated ApoB Crook siRNA were compared with the control treatment group administered with saline. Statistical analysis was applied using the two-tailed paired T test algorithm. Results show a substantive reduction in mean plasma ApoB levels in mice treated with GalNAc-conjugated Crook siRNA, compared to control. However, it just fails significance (p=0.08), most likely due to small sample size and variation in ApoB levels between control animals.

With reference to FIG. 1 (b), plasma ApoB levels (micrograms/ml) measured 96 hours following administration of GalNAc-conjugated ApoB Crook siRNA were compared to the control group, treated with siRNA construct unconjugated (without GalNAc) ApoB Crook siRNA. Statistical analysis was applied using the two-tailed paired T test algorithm. Results show a highly significant reduction in plasma ApoB levels in this GalNAc-conjugated Crook siRNA treatment group when compared to control unconjugated siRNA with Crook (P=0.00435832).

### EXAMPLE 2

FIG. 2a-c compares the relative silencing activities of 20 PCSK9 crook siRNAs in vitro. HepG2 cells were reverse transfected with a library of 20 custom crook siRNAs (10 sense siRNAs and 10 antisense siRNAs) alongside the siRNA controls using conditions identified in the assay development phase. A five-point dose range (100 nM, 25 nM, 6.25 nM, 1.56 nM and 0.39 nM) was used with four replicates per siRNA concentration.

72 h post transfection, PCSK9 mRNA levels were quantified by duplex RT-qPCR, normalising to housekeeping reference gene GAPDH, and then to the average expression of PCSK9 across the five doses of the corresponding negative (NEG) crook siRNA control (Sense or Antisense).

Most siRNAs induce some PCSK9 mRNA decrease, however with various efficiency; see FIG. 2a-c. PCSK9 mRNA levels tend to increase at high siRNA concentrations (>6.25 nM for sense and >25 nM for antisense). The optimal concentration is 6.25 nM for sense siRNAs and 25 nM for antisense siRNAs.; see FIG. 3.

In conclusion 4 crook siRNAs have efficiency >80% (sense siRNAs PC8, PC9, PC10 and antisense siRNA PC18) at optimal concentration; see table 4 below.

TABLE 4
Sense and antisense pairing. The nucleic acid
molecules in each row e.g., SEQ ID NO 1 and 11
are complementary and hybridise forming a double
stranded RNA. The pair can either comprise a
crook sequence on the sense or antisense sequence.
Thus, each combination of sense and antisense
forms two different nucleic acid molecules e.g.,
SEQ ID NO 1 and 11 wherein i) the sense sequence
comprises the crook or ii) wherein the antisense
sequence comprises the crook.

| NAME | | | | | |
|---|---|---|---|---|---|
| Sense | antisense | | SEQ ID | | SEQ ID |
| crook | crook | Sense Sequence | NO | Antisense Sequence | NO |
| PC01 | PC11 | 5′- | 1 | 5′- | 11 |
| | | CCUCAUAGGCCUGGAGU | | AUAAACUCCAGGCCUAUG | |
| | | UUAU-3′ | | AGG-3′ | |
| PC02 | PC12 | 5′- | 2 | 5′- | 12 |
| | | AGGCCUGGAGUUUAUUC | | UUCCGAAUAAACUCCAGG | |
| | | GGAA-3′ | | CCU-3′ | |
| PC03 | PC13 | 5′- | 3 | 5′- | 13 |
| | | CCCUCAUAGGCCUGGAG | | UAAACUCCAGGCCUAUGA | |
| | | UUUA-3′ | | GGG-3′ | |
| PC04 | PC14 | 5′- | 4 | 5′- | 14 |
| | | ACCCUCAUAGGCCUGGA | | AAACUCCAGGCCUAUGAG | |
| | | GUUU-3′ | | GGU-3′ | |
| PC05 | PC15 | 5′- | 5 | 5′- | 15 |
| | | UAGGCCUGGAGUUUAUU | | UCCGAAUAAACUCCAGGC | |
| | | CGGA-3′ | | CUA-3′ | |
| PC06 | PC16 | 5′- | 6 | 5′- | 16 |
| | | AGGUCUGGAAUGCAAAG | | UUGACUUUGCAUUCCAGA | |
| | | UCAA-3′ | | CCU-3′ | |
| PC07 | PC17 | 5′- | 7 | 5′- | 17 |
| | | GGCCUGGAGUUUAUUCG | | UUUCCGAAUAAACUCCAG | |
| | | GAAA-3′ | | GCC-3′ | |
| PC08 | PC18 | 5′- | 8 | 5′- | 18 |
| | | CAGGUCUGGAAUGCAAA | | UGACUUUGCAUUCCAGAC | |
| | | GUCA-3′* | | CUG-3′ | |
| PC09 | PC19 | 5′- | 9 | 5′- | 19 |
| | | CCUCACCAAGAUCCUGC | | ACAUGCAGGAUCUUGGUG | |
| | | AUGU-3′ | | AGG-3′ | |
| PC10 | PC20 | 5′- | 10 | 5′- | 20 |
| | | CACCAGCAUACAGAGUG | | UGGUCACUCUGUAUGCUG | |
| | | ACCA-3′ | | GUG-3′ | |
| PC21 | PC77 | 5′- | 21 | 5′- | 77 |
| | | AGCAAGCAGACAUUUAU | | AAAGAUAAAUGUCUGCUU | |
| | | CUUU-3′ | | GCU-3′ | |
| PC22 | PC78 | 5′- | 22 | 5′- | 78 |
| | | AGGUCUGGAAUGCAAAG | | UUGACUUUGCAUUCCAGA | |
| | | UCAA-3′ | | CCU-3′ | |
| PC23 | PC79 | 5′- | 23 | 5′- | 79 |
| | | GGCCUGGAGUUUAUUCG | | UUUCCGAAUAAACUCCAG | |
| | | GAAA-3′ | | GCC-3′ | |
| PC24 | PC80 | 5′- | 24 | 5′- | 80 |
| | | CAGGUCUGGAAUGCAAA | | UGACUUUGCAUUCCAGAC | |
| | | GUCA-3′ | | CUG-3′ | |
| PC25 | PC81 | 5′- | 25 | 5′- | 81 |
| | | CCCAAGCAAGCAGACAU | | AUAAAUGUCUGCUUGCUU | |
| | | UUAU-3′ | | GGG-3′ | |
| PC26 | PC82 | 5′- | 26 | 5′- | 82 |
| | | CCUCACCAAGAUCCUGC | | ACAUGCAGGAUCUUGGUG | |
| | | AUGU-3′ | | AGG-3′ | |
| PC27 | PC83 | 5′- | 27 | 5′- | 83 |
| | | UUUUCUAGACCUGUUUU | | AAGCAAAACAGGUCUAGA | |
| | | GCUU-3′ | | AAA-3′ | |
| PC28 | PC84 | 5′- | 28 | 5′- | 84 |
| | | ACCCAAGCAAGCAGACA | | UAAAUGUCUGCUUGCUUG | |
| | | UUUA-3′ | | GGU-3′ | |
| PC29 | PC85 | 5′- | 29 | 5′- | 85 |
| | | CACCAGCAUACAGAGUG | | UGGUCACUCUGUAUGCUG | |
| | | ACCA-3′ | | GUG-3′ | |
| PC30 | PC86 | 5′- | 30 | 5′- | 86 |
| | | AUUCUGGGUUUUGUAGC | | AAAUGCUACAAAACCCAG | |
| | | AUUU-3′ | | AAU-3′ | |
| PC31 | PC87 | 5′- | 31 | 5′- | 87 |
| | | AUCUCCUAGACACCAGC | | GUAUGCUGGUGUCUAGGA | |
| | | AUAC-3′ | | GAU-3′ | |
| PC32 | PC88 | 5′- | 32 | 5′- | 88 |
| | | UCCUAGACACCAGCAUA | | UCUGUAUGCUGGUGUCUA | |
| | | CAGA-3′ | | GGA-3′ | |
| PC33 | PC89 | 5′- | 33 | 5′- | 89 |
| | | GACAUUUAUCUUUUGGG | | CAGACCCAAAAGAUAAAU | |
| | | UCUG-3′ | | GUC-3′ | |
| PC34 | PC90 | 5′- | 34 | 5′- | 90 |
| | | UAUUCUGGGUUUUGUAG | | AAUGCUACAAAACCCAGA | |
| | | CAUU-3′ | | AUA-3′ | |
| PC35 | PC91 | 5′- | 35 | 5′- | 91 |
| | | CUGGAGUUUAUUCGGAA | | GCUUUUCCGAAUAAACUC | |
| | | AAGC-3′ | | CAG-3′ | |
| PC36 | PC92 | 5′- | 36 | 5′- | 92 |
| | | GCCUGGAGUUUAUUCGG | | UUUUCCGAAUAAACUCCA | |
| | | AAAA-3′ | | GGC-3′ | |
| PC37 | PC93 | 5′- | 37 | 5′- | 93 |
| | | GAGGCAGAGACUGAUCC | | AAGUGGAUCAGUCUCUGC | |
| | | ACUU-3′ | | CUC-3′ | |
| PC38 | PC94 | 5′- | 38 | 5′- | 94 |
| | | AAGCAAGCAGACAUUUA | | AAGAUAAAUGUCUGCUUG | |
| | | UCUU-3′ | | CUU-3′ | |
| PC39 | PC95 | 5′- | 39 | 5′- | 95 |
| | | UAGACCUGUUUUGCUUU | | UACAAAAGCAAAACAGGU | |
| | | UGUA-3′ | | CUA-3′ | |
| PC40 | PC96 | 5′- | 40 | 5′- | 96 |
| | | UUUGCUUUUGUAACUUG | | UCUUCAAGUUACAAAAGC | |
| | | AAGA-3′ | | AAA-3′ | |
| PC41 | PC97 | 5′- | 41 | 5′- | 97 |
| | | CACUUCUCUGCCAAAGA | | GACAUCUUUGGCAGAGAA | |
| | | UGUC-3′ | | GUG-3′ | |
| PC42 | PC98 | 5′- | 42 | 5′- | 98 |
| | | UUGCUUUUGUAACUUGA | | AUCUUCAAGUUACAAAAG | |
| | | AGAU-3′ | | CAA-3′ | |
| PC43 | PC99 | 5′- | 43 | 5′- | 99 |
| | | AUGCAAAGUCAAGGAGC | | CCAUGCUCCUUGACUUUG | |
| | | AUGG-3′ | | CAU-3′ | |
| PC44 | PC100 | 5′- | 44 | 5′- | 100 |
| | | CCCACCCAAGCAAGCAG | | AUGUCUGCUUGCUUGGGU | |
| | | ACAU-3′ | | GGG-3′ | |
| PC45 | PC101 | 5′- | 45 | 5′- | 101 |
| | | GGGUAACAGUGAGGCUG | | UUCCCAGCCUCACUGUUA | |
| | | GGAA-3′ | | CCC-3′ | |
| PC46 | PC102 | 5′- | 46 | 5′- | 102 |
| | | GGUCAUGGUCACCGACU | | UCGAAGUCGGUGACCAUG | |
| | | UCGA-3′ | | ACC-3′ | |
| PC47 | PC103 | 5′- | 47 | 5′- | 103 |
| | | GGCAGCUGUUUUGCAGG | | CAGUCCUGCAAAACAGCU | |
| | | ACUG-3′ | | GCC-3′ | |
| PC48 | PC104 | 5′- | 48 | 5′- | 104 |
| | | GGGCAGGUUGGCAGCUG | | AAAACAGCUGCCAACCUG | |
| | | UUUU-3′ | | CCC-3′ | |
| PC49 | PC105 | 5′- | 49 | 5′- | 105 |
| | | UUGAAGAUAUUUAUUCU | | ACCCAGAAUAAAUAUCUU | |
| | | GGGU-3′ | | CAA-3′ | |
| PC50 | PC106 | 5′- | 50 | 5′- | 106 |
| | | UGGCAGCUGUUUUGCAG | | AGUCCUGCAAAACAGCUG | |
| | | GACU-3′ | | CCA-3′ | |
| PC51 | PC107 | 5′- | 51 | 5′- | 107 |
| | | CCGGGGAUACCUCACCA | | AUCUUGGUGAGGUAUCCC | |
| | | AGAU-3′ | | CGG-3′ | |
| PC52 | PC108 | 5′- | 52 | 5′- | 108 |
| | | ACUGAUCCACUUCUCUG | | UUGGCAGAGAAGUGGAUC | |
| | | CCAA-3′ | | AGU-3′ | |
| PC53 | PC109 | 5′- | 53 | 5′- | 109 |
| | | AUCCACUUCUCUGCCAA | | AUCUUUGGCAGAGAAGUG | |
| | | AGAU-3′ | | GAU-3′ | |
| PC54 | PC110 | 5′- | 54 | 5′- | 110 |
| | | ACUUCUCUGCCAAAGAU | | UGACAUCUUUGGCAGAGA | |
| | | GUCA-3′ | | AGU-3′ | |
| PC55 | PC111 | 5′- | 55 | 5′- | 111 |
| | | GUCUGGAAUGCAAAGUC | | CCUUGACUUUGCAUUCCA | |
| | | AAGG-3′ | | GAC-3′ | |
| PC56 | PC112 | 5′- | 56 | 5′- | 112 |
| | | CUUCUCUGCCAAAGAUG | | AUGACAUCUUUGGCAGAG | |
| | | UCAU-3′ | | AAG-3′ | |
| PC57 | PC113 | 5′- | 57 | 5′- | 113 |
| | | GAGUUGAGGCAGAGACU | | GAUCAGUCUCUGCCUCAA | |
| | | GAUC-3′ | | CUC-3′ | |
| PC58 | PC114 | 5′- | 58 | 5′- | 114 |
| | | GACCUGUUUUGCUUUUG | | GUUACAAAAGCAAAACAG | |
| | | UAAC-3′ | | GUC-3′ | |
| PC59 | PC115 | 5′- | 59 | 5′- | 115 |
| | | CGGGGAUACCUCACCAA | | GAUCUUGGUGAGGUAUCC | |
| | | GAUC-3′ | | CCG-3′ | |
| PC60 | PC116 | 5′- | 60 | 5′- | 116 |
| | | UUUCUAGACCUGUUUUG | | AAAGCAAAACAGGUCUAG | |
| | | CUUU-3′ | | AAA-3′ | |
| PC61 | PC117 | 5′- | 61 | 5′- | 117 |
| | | GGUCUGGAAUGCAAAGU | | CUUGACUUUGCAUUCCAG | |
| | | CAAG-3′ | | ACC-3′ | |
| PC62 | PC118 | 5′- | 62 | 5′- | 118 |
| | | UAUCUCCUAGACACCAG | | UAUGCUGGUGUCUAGGAG | |
| | | CAUA-3′ | | AUA-3′ | |
| PC63 | PC119 | 5′- | 63 | 5′- | 119 |
| | | AGGUUGGCAGCUGUUUU | | CUGCAAAACAGCUGCCAA | |
| | | GCAG-3′ | | CCU-3′ | |
| PC64 | PC120 | 5′- | 64 | 5′- | 120 |
| | | AACUUUUCUAGACCUGU | | CAAAACAGGUCUAGAAAA | |
| | | UUUG-3′ | | GUU-3′ | |
| PC65 | PC121 | 5′- | 65 | 5′- | 121 |
| | | CUUUUCUAGACCUGUUU | | AGCAAAACAGGUCUAGAA | |
| | | UGCU-3′ | | AAG-3′ | |
| PC66 | PC122 | 5′- | 66 | 5′- | 122 |
| | | UCCACUUCUCUGCCAAA | | CAUCUUUGGCAGAGAAGU | |
| | | GAUG-3′ | | GGA-3′ | |
| PC67 | PC123 | 5′- | 67 | 5′- | 123 |
| | | UGGAGUUUAUUCGGAAA | | GGCUUUUCCGAAUAAACU | |
| | | AGCC-3′ | | CCA-3′ | |
| PC68 | PC124 | 5′- | 68 | 5′- | 124 |
| | | GGCAGGUUGGCAGCUGU | | CAAAACAGCUGCCAACCU | |
| | | UUUG-3′ | | GCC-3′ | |
| PC69 | PC125 | 5′- | 69 | 5′- | 125 |
| | | UGGAGGUGUAUCUCCUA | | UGUCUAGGAGAUACACCU | |
| | | GACA-3′ | | CCA-3′ | |
| PC70 | PC126 | 5′- | 70 | 5′- | 126 |
| | | GUCAUCAAUGAGGCCUG | | GAACCAGGCCUCAUUGAU | |
| | | GUUC-3′ | | GAC-3′ | |
| PC71 | PC127 | 5′- | 71 | 5′- | 127 |
| | | UUCUAGACCUGUUUUGC | | AAAAGCAAAACAGGUCUA | |
| | | UUUU-3′ | | GAA-3′ | |
| PC72 | PC128 | 5′- | 72 | 5′- | 128 |
| | | UUCUGGGUUUUGUAGCA | | AAAAUGCUACAAAACCCA | |
| | | UUUU-3′ | | GAA-3′ | |
| PC73 | PC129 | 5′- | 73 | 5′- | 129 |
| | | GAGACUGAUCCACUUCU | | GCAGAGAAGUGGAUCAGU | |
| | | CUGC-3′ | | CUC-3′ | |
| PC74 | PC130 | 5′- | 74 | 5′- | 130 |
| | | AGUCAAGGAGCAUGGAA | | GGGAUUCCAUGCUCCUUG | |
| | | UCCC-3′ | | ACU-3′ | |
| PC75 | PC131 | 5′- | 75 | 5′- | 131 |
| | | AUCUUUUGGGUCUGUCC | | GAGAGGACAGACCCAAAA | |
| | | UCUC-3′ | | GAU-3′ | |
| PC76 | PC132 | 5′- | 76 | 5′- | 132 |
| | | CACCCAAGCAAGCAGAC | | AAAUGUCUGCUUGCUUGG | |
| | | AUUU-3′ | | GUG-3′ | |

### REFERENCES

Nair, J. K., Willoughby, J. L., Chan, A., Charisse, K., Alam, M. R., Wang, a Hoekstra, M., Kandasamy, P., Kel'in, A. V,, Milstein, S. and Taneja, N,, 2014. Multivalent N-acetylgalactosamine-conjugated siRNA localizes in hepatocytes and elicits robust RNAi-mediated gene silencing. Journal of the American Chemical Society, 136(49), pp. 16958-16961.
Soutschek, J., Akinc, A., Bramlage, B., Charisse, K., Constien, R., Donoghue, M,, Elbashir, S., Geick, A., Hadwiger, P., Harborth, J. and John, M., 2004. Therapeutic silencing of an endogenous gene by systemic administration of modified siRNAs. Nature, 432(7014), p. 173.
A D Judge, V Sood, J R Shaw, D Fang, K McClintock, I MacLachlan. Sequence-dependent stimulation of the mammalian innate immune response by synthetic siRNA. Nat Biotechnol 2005. 23(4):457-62.
A D Judge, G Bola, A Lee, I MacLachlan. Design of noninflammatory synthetic siRNA mediating potent gene silencing in vivo. Mol Ther 2006. 13(3):494-505.
S J Allison, J Milner. RNA Interference by Single- and double-stranded siRNA with a DNA extension containing a 3′ nuclease-resistant mini-hairpin structure. Mol Ther Nucleic Acids 2014. 7; 2(1):e141.

1. A nucleic acid molecule comprising:
a first part that comprises a double stranded inhibitory ribonucleic acid (RNA) molecule comprising a sense strand and an antisense strand of at least part of the human PCSK9 nucleotide sequence or polymorphic sequence variant thereof; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, wherein said single stranded DNA molecule comprises a nucleotide sequence that is adapted over at least part of its length to anneal by complementary base pairing to a part of said single stranded DNA to form a double stranded DNA structure comprising a double stranded stem domain and a single stranded loop domain.

2. The nucleic acid molecule according to claim 1 wherein:
the 5′ end of said single stranded DNA molecule is covalently linked to the 3′ end of the sense strand of the double stranded inhibitory RNA molecule; or
the 5′ end of said single stranded DNA molecule is covalently linked to the 3′ end of the antisense strand of the double stranded inhibitory RNA molecule.

3. (canceled)

4. The nucleic acid molecule according to claim 1 wherein said loop portion comprises a region comprising the nucleotide sequence GNA or GNNA, wherein each N independently represents guanine (G), thymidine (T), adenine (A), or cytosine (C).

5. The nucleic acid molecule according to claim 4 wherein said loop domain comprises the nucleotide sequence GCGAAGC.

6. The nucleic acid molecule according to claim 1 wherein said single stranded DNA molecule comprises the nucleotide sequence TCACCTCATCCCGCGAAGC (SEQ ID NO: 133).

7. The nucleic acid molecule according to claim 1 wherein:
said double stranded inhibitory RNA molecule is between 18 and 29 nucleotide base pairs in length, more preferably between 19 and 23 nucleotide base pairs in length;
said double stranded inhibitory RNA molecule comprises or consists of between 18 and 29 contiguous nucleotides of the sense nucleotide sequence set forth in SEQ ID NO: 134; or
said double stranded inhibitory RNA molecule comprises or consists of 21 contiguous nucleotide bases pairs of the sense nucleotide sequence set forth in SEQ ID NO: 134.

8-9. (canceled)

10. The nucleic acid molecule according to claim 1 wherein:
said double stranded inhibitory RNA molecule comprises a sense nucleotide sequence selected from the group consisting of: SEQ ID NO: 8, 1, 2, 3, 4, 5, 6, 7, 9 or 10;
said double stranded inhibitory RNA molecule comprises an antisense nucleotide sequence selected from the group consisting of: SEQ ID NO: 18, 11, 12, 13, 14, 15, 16, 17, 19 or 20;
said double stranded inhibitory RNA molecule comprises a sense nucleotide sequence selected from the group consisting of: SEQ ID NO: 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 and 76; or
said double stranded inhibitory RNA molecule comprises an antisense nucleotide sequence selected from the group consisting of: SEQ ID NO: 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131 and 132.

11-13. (canceled)

14. The nucleic acid molecule according to claim 1 wherein:
said double stranded inhibitory RNA molecule comprises a sense strand comprising SEQ ID NO: 8 and an antisense strand comprising SEQ ID NO: 18;
said double stranded inhibitory RNA molecule comprises a sense strand comprising SEQ ID NO: 9 and an antisense strand comprising SEQ ID NO: 19;
said double stranded inhibitory RNA molecule comprises a sense strand comprising SEQ ID NO: 10 and an antisense strand comprising SEQ ID NO: 20; or
said double stranded inhibitory RNA molecule comprises a sense strand comprising SEQ ID NO: 135 and an antisense strand comprising SEQ ID NO: 136.

15. The nucleic acid molecule according to claim 14 wherein:
said single stranded DNA molecule is covalently linked to a sense strand comprising SEQ ID NO: 8; or
single stranded DNA molecule is covalently linked to an antisense strand comprising SEQ ID NO: 18.

16-17. (canceled)

18. The nucleic acid molecule according to claim 14 wherein:
said single stranded DNA molecule is covalently linked to a sense strand comprising SEQ ID NO: 9; or
single stranded DNA molecule is covalently linked to an antisense strand comprising SEQ ID NO: 19.

19-20. (canceled)

21. The nucleic acid molecule according to claim 14 wherein:
said single stranded DNA molecule is covalently linked to a sense strand comprising SEQ ID NO: 10; or
said single stranded DNA molecule is covalently linked to an antisense strand comprising SEQ ID NO: 20.

22-23. (canceled)

24. The nucleic acid molecule according to claim 1 wherein:
N-acetylgalactosamine is linked to the DNA part of said nucleic acid molecule via a terminal 3′ end of the DNA part; or
N-acetylgalactosamine is linked to the either the antisense part of said inhibitory RNA or the sense part of said inhibitory RNA.

25. (canceled)

26. The nucleic acid molecule according to claim 24 wherein N-acetylgalactosamine comprises the structure:

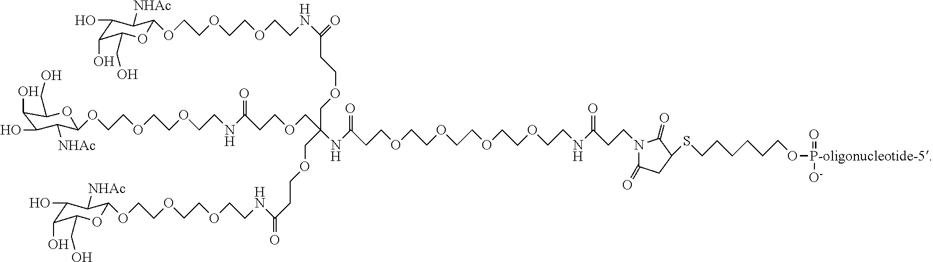

27. A pharmaceutical composition comprising at least one nucleic acid molecule according to claim 1 and a pharmaceutical carrier and/or excipient.

28. The pharmaceutical composition according to claim 27 wherein said composition comprises at least one further, different, therapeutic agent.

29. The pharmaceutical composition according to claim 28 wherein said further therapeutic agent is a statin.

30-34. (canceled)

35. A method to treat a subject that has or is predisposed to hypercholesterolemia comprising administering an effective dose of a nucleic acid according to claim 1, or a pharmaceutical composition thereof, thereby treating or preventing hypercholesterolemia or a disease associated with hypercholesterolemia.

36. The method according to claim 35 wherein the hypercholesterolemia is familial hypercholesterolemia and/or said subject is resistant to statin therapy.

37. The method according to claim 36 wherein familial hypercholesterolemia is associated with elevated levels of PCSK9 expression.

38. (canceled)

39. The method according to claim 35 wherein said disease associated with hypercholesterolemia is selected from the group consisting of: stroke prevention, hyperlipidaemia, cardiovascular disease, atherosclerosis, coronary heart disease, cerebrovascular disease, peripheral arterial disease, hypertension, metabolic syndrome, type II diabetes, non-alcoholic fatty acid liver disease and non-alcoholic steatohepatitis.


SEQUENCE LISTING (145 sequences)

SEQ ID NO: 1 - RNA, 21 nt, artificial sequence
  sense/antisense RNA
ccucauaggccuggaguuuau

SEQ ID NO: 2 - RNA, 21 nt, artificial sequence
  sense/antisense RNA
aggccuggaguuuauucggaa

SEQ ID NO: 3 - RNA, 21 nt, artificial sequence
  sense/antisense RNA
cccucauaggccuggaguuua

SEQ ID NO: 4 - RNA, 21 nt, artificial sequence
  sense/antisense RNA
acccucauaggccuggaguuu

SEQ ID NO: 5 - RNA, 21 nt, artificial sequence
  sense/antisense RNA
uaggccuggaguuuauucgga

SEQ ID NO: 6 - RNA, 21 nt, artificial sequence
  sense/antisense RNA
aggucuggaaugcaaagucaa

SEQ ID NO: 7 - RNA, 21 nt, artificial sequence
  sense/antisense RNA
ggccuggaguuuauucggaaa

SEQ ID NO: 8 - RNA, 21 nt, artificial sequence
  sense/antisense RNA
caggucuggaaugcaaaguca

SEQ ID NO: 9 - RNA, 21 nt, artificial sequence
  sense/antisense RNA
ccucaccaagauccugcaugu

SEQ ID NO: 10 - RNA, 21 nt, artificial sequence
  sense/antisense RNA
caccagcauacagagugacca

SEQ ID NO: 11 - RNA, 21 nt, artificial sequence
  sense/antisense RNA
auaaacuccaggccuaugagg

SEQ ID NO: 12 - RNA, 21 nt, artificial sequence
  sense/antisense RNA
uuccgaauaaacuccaggccu

SEQ ID NO: 13 - RNA, 21 nt, artificial sequence
  sense/antisense RNA
uaaacuccaggccuaugaggg

SEQ ID NO: 14 - RNA, 21 nt, artificial sequence
  sense/antisense RNA
aaacuccaggccuaugagggu

SEQ ID NO: 15 - RNA, 21 nt, artificial sequence
  sense/antisense RNA
uccgaauaaacuccaggccua

SEQ ID NO: 16 - RNA, 21 nt, artificial sequence
  sense/antisense RNA
uugacuuugcauuccagaccu

SEQ ID NO: 17 - RNA, 21 nt, artificial sequence
  sense/antisense RNA
uuuccgaauaaacuccaggcc

SEQ ID NO: 18 - RNA, 21 nt, artificial sequence
  sense/antisense RNA
ugacuuugcauuccagaccug

SEQ ID NO: 19 - RNA, 21 nt, artificial sequence
  sense/antisense RNA
acaugcaggaucuuggugagg

SEQ ID NO: 20 - RNA, 21 nt, artificial sequence
  sense/antisense RNA
uggucacucuguaugcuggug

SEQ ID NO: 21 - RNA, 21 nt, artificial sequence
  sense/antisense RNA
agcaagcagacauuuaucuuu

SEQ ID NO: 22 - RNA, 21 nt, artificial sequence
  sense/antisense RNA
aggucuggaaugcaaagucaa

SEQ ID NO: 23 - RNA, 21 nt, artificial sequence
  sense/antisense RNA
ggccuggaguuuauucggaaa

SEQ ID NO: 24 - RNA, 21 nt, artificial sequence
  sense/antisense RNA
caggucuggaaugcaaaguca

SEQ ID NO: 25 - RNA, 21 nt, artificial sequence
  sense/antisense RNA
cccaagcaagcagacauuuau

SEQ ID NO: 26 - RNA, 21 nt, artificial sequence
  sense/antisense RNA
ccucaccaagauccugcaugu

SEQ ID NO: 27 - RNA, 21 nt, artificial sequence
  sense/antisense RNA
uuuucuagaccuguuuugcuu

SEQ ID NO: 28 - RNA, 21 nt, artificial sequence
  sense/antisense RNA
acccaagcaagcagacauuua

SEQ ID NO: 29 - RNA, 21 nt, artificial sequence
  sense/antisense RNA
caccagcauacagagugacca

SEQ ID NO: 30 - RNA, 21 nt, artificial sequence
  sense/antisense RNA
auucuggguuuuguagcauuu

SEQ ID NO: 31 - RNA, 21 nt, artificial sequence
  sense/antisense RNA
aucuccuagacaccagcauac

SEQ ID NO: 32 - RNA, 21 nt, artificial sequence
  sense/antisense RNA
uccuagacaccagcauacaga

SEQ ID NO: 33 - RNA, 21 nt, artificial sequence
  sense/antisense RNA
gacauuuaucuuuugggucug

SEQ ID NO: 34 - RNA, 21 nt, artificial sequence
  sense/antisense RNA
uauucuggguuuuguagcauu

SEQ ID NO: 35 - RNA, 21 nt, artificial sequence
  sense/antisense RNA
cuggaguuuauucggaaaagc

SEQ ID NO: 36 - RNA, 21 nt, artificial sequence
  sense/antisense RNA
gccuggaguuuauucggaaaa

SEQ ID NO: 37 - RNA, 21 nt, artificial sequence
  sense/antisense RNA
gaggcagagacugauccacuu

SEQ ID NO: 38 - RNA, 21 nt, artificial sequence
  sense/antisense RNA
aagcaagcagacauuuaucuu

SEQ ID NO: 39 - RNA, 21 nt, artificial sequence
  sense/antisense RNA
uagaccuguuuugcuuuugua

SEQ ID NO: 40 - RNA, 21 nt, artificial sequence
  sense/antisense RNA
uuugcuuuuguaacuugaaga

SEQ ID NO: 41 - RNA, 21 nt, artificial sequence
  sense/antisense RNA
cacuucucugccaaagauguc

SEQ ID NO: 42 - RNA, 21 nt, artificial sequence
  sense/antisense RNA
uugcuuuuguaacuugaagau

SEQ ID NO: 43 - RNA, 21 nt, artificial sequence
  sense/antisense RNA
augcaaagucaaggagcaugg

SEQ ID NO: 44 - RNA, 21 nt, artificial sequence
  sense/antisense RNA
cccacccaagcaagcagacau

SEQ ID NO: 45 - RNA, 21 nt, artificial sequence
  sense/antisense RNA
ggguaacagugaggcugggaa

SEQ ID NO: 46 - RNA, 21 nt, artificial sequence
  sense/antisense RNA
ggucauggucaccgacuucga

SEQ ID NO: 47 - RNA, 21 nt, artificial sequence
  sense/antisense RNA
ggcagcuguuuugcaggacug

SEQ ID NO: 48 - RNA, 21 nt, artificial sequence
  sense/antisense RNA
gggcagguuggcagcuguuuu

SEQ ID NO: 49 - RNA, 21 nt, artificial sequence
  sense/antisense RNA
uugaagauauuuauucugggu

SEQ ID NO: 50 - RNA, 21 nt, artificial sequence
  sense/antisense RNA
uggcagcuguuuugcaggacu

SEQ ID NO: 51 - RNA, 21 nt, artificial sequence
  sense/antisense RNA
ccggggauaccucaccaagau

SEQ ID NO: 52 - RNA, 21 nt, artificial sequence
  sense/antisense RNA
acugauccacuucucugccaa

SEQ ID NO: 53 - RNA, 21 nt, artificial sequence
  sense/antisense RNA
auccacuucucugccaaagau

SEQ ID NO: 54 - RNA, 21 nt, artificial sequence
  sense/antisense RNA
acuucucugccaaagauguca

SEQ ID NO: 55 - RNA, 21 nt, artificial sequence
  sense/antisense RNA
gucuggaaugcaaagucaagg

SEQ ID NO: 56 - RNA, 21 nt, artificial sequence
  sense/antisense RNA
cuucucugccaaagaugucau

SEQ ID NO: 57 - RNA, 21 nt, artificial sequence
  sense/antisense RNA
gaguugaggcagagacugauc

SEQ ID NO: 58 - RNA, 21 nt, artificial sequence
  sense/antisense RNA
gaccuguuuugcuuuuguaac

SEQ ID NO: 59 - RNA, 21 nt, artificial sequence
  sense/antisense RNA
cggggauaccucaccaagauc

SEQ ID NO: 60 - RNA, 21 nt, artificial sequence
  sense/antisense RNA
uuucuagaccuguuuugcuuu

SEQ ID NO: 61 - RNA, 21 nt, artificial sequence
  sense/antisense RNA
ggucuggaaugcaaagucaag

SEQ ID NO: 62 - RNA, 21 nt, artificial sequence
  sense/antisense RNA
uaucuccuagacaccagcaua

SEQ ID NO: 63 - RNA, 21 nt, artificial sequence
  sense/antisense RNA
agguuggcagcuguuuugcag

SEQ ID NO: 64 - RNA, 21 nt, artificial sequence
  sense/antisense RNA
aacuuuucuagaccuguuuug

SEQ ID NO: 65 - RNA, 21 nt, artificial sequence
  sense/antisense RNA
cuuuucuagaccuguuuugcu

SEQ ID NO: 66 - RNA, 21 nt, artificial sequence
  sense/antisense RNA
uccacuucucugccaaagaug

SEQ ID NO: 67 - RNA, 21 nt, artificial sequence
  sense/antisense RNA
uggaguuuauucggaaaagcc

SEQ ID NO: 68 - RNA, 21 nt, artificial sequence
  sense/antisense RNA
ggcagguuggcagcuguuuug

SEQ ID NO: 69 - RNA, 21 nt, artificial sequence
  sense/antisense RNA
uggagguguaucuccuagaca

SEQ ID NO: 70 - RNA, 21 nt, artificial sequence
  sense/antisense RNA
gucaucaaugaggccugguuc

SEQ ID NO: 71 - RNA, 21 nt, artificial sequence
  sense/antisense RNA
uucuagaccuguuuugcuuuu

SEQ ID NO: 72 - RNA, 21 nt, artificial sequence
  sense/antisense RNA
uucuggguuuuguagcauuuu

SEQ ID NO: 73 - RNA, 21 nt, artificial sequence
  sense/antisense RNA
gagacugauccacuucucugc

SEQ ID NO: 74 - RNA, 21 nt, artificial sequence
  sense/antisense RNA
agucaaggagcauggaauccc

SEQ ID NO: 75 - RNA, 21 nt, artificial sequence
  sense/antisense RNA
aucuuuugggucuguccucuc

SEQ ID NO: 76 - RNA, 21 nt, artificial sequence
  sense/antisense RNA
cacccaagcaagcagacauuu

SEQ ID NO: 77 - RNA, 21 nt, artificial sequence
  sense/antisense RNA
aaagauaaaugucugcuugcu

SEQ ID NO: 78 - RNA, 21 nt, artificial sequence
  sense/antisense RNA
uugacuuugcauuccagaccu

SEQ ID NO: 79 - RNA, 21 nt, artificial sequence
  sense/antisense RNA
uuuccgaauaaacuccaggcc

SEQ ID NO: 80 - RNA, 21 nt, artificial sequence
  sense/antisense RNA
ugacuuugcauuccagaccug

SEQ ID NO: 81 - RNA, 21 nt, artificial sequence
  sense/antisense RNA
auaaaugucugcuugcuuggg

SEQ ID NO: 82 - RNA, 21 nt, artificial sequence
  sense/antisense RNA
acaugcaggaucuuggugagg

SEQ ID NO: 83 - RNA, 21 nt, artificial sequence
  sense/antisense RNA
aagcaaaacaggucuagaaaa

SEQ ID NO: 84 - RNA, 21 nt, artificial sequence
  sense/antisense RNA
uaaaugucugcuugcuugggu

SEQ ID NO: 85 - RNA, 21 nt, artificial sequence
  sense/antisense RNA
uggucacucuguaugcuggug

SEQ ID NO: 86 - RNA, 21 nt, artificial sequence
  sense/antisense RNA
aaaugcuacaaaacccagaau

SEQ ID NO: 87 - RNA, 21 nt, artificial sequence
  sense/antisense RNA
guaugcuggugucuaggagau

SEQ ID NO: 88 - RNA, 21 nt, artificial sequence
  sense/antisense RNA
ucuguaugcuggugucuagga

SEQ ID NO: 89 - RNA, 21 nt, artificial sequence
  sense/antisense RNA
cagacccaaaagauaaauguc

SEQ ID NO: 90 - RNA, 21 nt, artificial sequence
  sense/antisense RNA
aaugcuacaaaacccagaaua

SEQ ID NO: 91 - RNA, 21 nt, artificial sequence
  sense/antisense RNA
gcuuuuccgaauaaacuccag

SEQ ID NO: 92 - RNA, 21 nt, artificial sequence
  sense/antisense RNA
uuuuccgaauaaacuccaggc

SEQ ID NO: 93 - RNA, 21 nt, artificial sequence
  sense/antisense RNA
aaguggaucagucucugccuc

SEQ ID NO: 94 - RNA, 21 nt, artificial sequence
  sense/antisense RNA
aagauaaaugucugcuugcuu

SEQ ID NO: 95 - RNA, 21 nt, artificial sequence
  sense/antisense RNA
uacaaaagcaaaacaggucua

SEQ ID NO: 96 - RNA, 21 nt, artificial sequence
  sense/antisense RNA
ucuucaaguuacaaaagcaaa

SEQ ID NO: 97 - RNA, 21 nt, artificial sequence
  sense/antisense RNA
gacaucuuuggcagagaagug

SEQ ID NO: 98 - RNA, 21 nt, artificial sequence
  sense/antisense RNA
aucuucaaguuacaaaagcaa

SEQ ID NO: 99 - RNA, 21 nt, artificial sequence
  sense/antisense RNA
ccaugcuccuugacuuugcau

SEQ ID NO: 100 - RNA, 21 nt, artificial sequence
  sense/antisense RNA
augucugcuugcuuggguggg

SEQ ID NO: 101 - RNA, 21 nt, artificial sequence
  sense/antisense RNA
uucccagccucacuguuaccc

SEQ ID NO: 102 - RNA, 21 nt, artificial sequence
  sense/antisense RNA
ucgaagucggugaccaugacc

SEQ ID NO: 103 - RNA, 21 nt, artificial sequence
  sense/antisense RNA
caguccugcaaaacagcugcc

SEQ ID NO: 104 - RNA, 21 nt, artificial sequence
  sense/antisense RNA
aaaacagcugccaaccugccc

SEQ ID NO: 105 - RNA, 21 nt, artificial sequence
  sense/antisense RNA
acccagaauaaauaucuucaa

SEQ ID NO: 106 - RNA, 21 nt, artificial sequence
  sense/antisense RNA
aguccugcaaaacagcugcca

SEQ ID NO: 107 - RNA, 21 nt, artificial sequence
  sense/antisense RNA
aucuuggugagguauccccgg

SEQ ID NO: 108 - RNA, 21 nt, artificial sequence
  sense/antisense RNA
uuggcagagaaguggaucagu

SEQ ID NO: 109 - RNA, 21 nt, artificial sequence
  sense/antisense RNA
aucuuuggcagagaaguggau

SEQ ID NO: 110 - RNA, 21 nt, artificial sequence
  sense/antisense RNA
ugacaucuuuggcagagaagu

SEQ ID NO: 111 - RNA, 21 nt, artificial sequence
  sense/antisense RNA
ccuugacuuugcauuccagac

SEQ ID NO: 112 - RNA, 21 nt, artificial sequence
  sense/antisense RNA
augacaucuuuggcagagaag

SEQ ID NO: 113 - RNA, 21 nt, artificial sequence
  sense/antisense RNA
gaucagucucugccucaacuc

SEQ ID NO: 114 - RNA, 21 nt, artificial sequence
  sense/antisense RNA
guuacaaaagcaaaacagguc

SEQ ID NO: 115 - RNA, 21 nt, artificial sequence
  sense/antisense RNA
gaucuuggugagguauccccg

SEQ ID NO: 116 - RNA, 21 nt, artificial sequence
  sense/antisense RNA
aaagcaaaacaggucuagaaa

SEQ ID NO: 117 - RNA, 21 nt, artificial sequence
  sense/antisense RNA
cuugacuuugcauuccagacc

SEQ ID NO: 118 - RNA, 21 nt, artificial sequence
  sense/antisense RNA
uaugcuggugucuaggagaua

SEQ ID NO: 119 - RNA, 21 nt, artificial sequence
  sense/antisense RNA
cugcaaaacagcugccaaccu

SEQ ID NO: 120 - RNA, 21 nt, artificial sequence
  sense/antisense RNA
caaaacaggucuagaaaaguu

SEQ ID NO: 121 - RNA, 21 nt, artificial sequence
  sense/antisense RNA
agcaaaacaggucuagaaaag

SEQ ID NO: 122 - RNA, 21 nt, artificial sequence
  sense/antisense RNA
caucuuuggcagagaagugga

SEQ ID NO: 123 - RNA, 21 nt, artificial sequence
  sense/antisense RNA
ggcuuuuccgaauaaacucca

SEQ ID NO: 124 - RNA, 21 nt, artificial sequence
  sense/antisense RNA
caaaacagcugccaaccugcc

SEQ ID NO: 125 - RNA, 21 nt, artificial sequence
  sense/antisense RNA
ugucuaggagauacaccucca

SEQ ID NO: 126 - RNA, 21 nt, artificial sequence
  sense/antisense RNA
gaaccaggccucauugaugac

SEQ ID NO: 127 - RNA, 21 nt, artificial sequence
  sense/antisense RNA
aaaagcaaaacaggucuagaa

SEQ ID NO: 128 - RNA, 21 nt, artificial sequence
  sense/antisense RNA
aaaaugcuacaaaacccagaa

SEQ ID NO: 129 - RNA, 21 nt, artificial sequence
  sense/antisense RNA
gcagagaaguggaucagucuc

SEQ ID NO: 130 - RNA, 21 nt, artificial sequence
  sense/antisense RNA
gggauuccaugcuccuugacu

SEQ ID NO: 131 - RNA, 21 nt, artificial sequence
  sense/antisense RNA
gagaggacagacccaaaagau

SEQ ID NO: 132 - RNA, 21 nt, artificial sequence
  sense/antisense RNA
aaaugucugcuugcuugggug

SEQ ID NO: 133 - DNA, 19 bp, artificial sequence
  crook sequence
tcacctcatcccgcgaagc

SEQ ID NO: 134 - DNA, 3637 bp, artificial sequence
  PCSK9 cDNA sequence
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa

SEQ ID NO: 135 - DNA, 21 bp, artificial sequence
  sense unmodified inclisiran; deoxythymidine (11)...(11);  DNA/RNA type sequence
cuagaccugutuugcuuuugu

SEQ ID NO: 136 - RNA, 23 nt, artificial sequence
  antisense unmodified inclisiran
acaaaagcaaaagaccucuagaa

SEQ ID NO: 137 - RNA, 21 nt, artificial sequence
  apoB-1 sense
gucaucacacugaauaccaau

SEQ ID NO: 138 - RNA, 23 nt, artificial sequence
  apoB-1 antisense
auugguauucagugugaugacac

SEQ ID NO: 139 - RNA, 21 nt, artificial sequence
  beta-Gal sense
uugauguguuuagucgcuauu

SEQ ID NO: 140 - RNA, 21 nt, artificial sequence
  beta-Gal antisense
uagcgacuaaacacaucaauu

SEQ ID NO: 141 - RNA, 19 nt, artificial sequence
  beta-gal 728 sense
cuacacaaaucagcgauuu

SEQ ID NO: 142 - RNA, 19 nt, artificial sequence
  beta-gal 728 antisense
aaaucgcugauuuguguag

SEQ ID NO: 143 - DNA, 21 bp, artificial sequence
  Luc-siRNA sense; deoxythymidine (20)..(21); DNA/RNA type sequence
uaaggcuaugaagagauactt

SEQ ID NO: 144 - RNA, 21 nt, artificial sequence
  Luc-siRNA antisense
aaguaucucuucauagccuua

SEQ ID NO: 145 - RNA, 23 nt, artificial sequence
  Antisense unmodified inclisiran
acaaaagcaaaacaggucuagaa